(19) United States
(12) Patent Application Publication
(10) Pub. No.: US 2009/0203661 A1
(43) Pub. Date: Aug. 13, 2009

(54) Betulinic acid, derivatives and analogs thereof and uses therefor

(76) Inventors: Stephen H. Safe, College Station, TX (US); Sudhakar Chintharlapalli, Indianapolis, IN (US)

Correspondence Address: Benjamin Aaron Adler;ADLER & ASSOCIATES, 8011 Candle Lane, Houston, TX, 77071, US

(21) Appl. No.: 11/974,405

(22) Filed: Oct. 12, 2007

Related U.S. Application Data
(60) Provisional application No. 60/851,113, filed on Oct. 12, 2006.

(51) Int. Cl. A61K 31/56; C07J 53/00; C12N 5/00

(52) U.S. Cl. 514/177; 552/510; 435/375

(57) ABSTRACT

Provided herein provided herein are novel analogs and derivatives of betulinic acid. Also provided is a method for inhibiting an activity of one or more specificity protein (Sp) transcription factors cells associated with a neoplastic disease using betulinic acid, betulinic acid analog(s) and/or derivative(s) effective to decrease expression of a microRNA with concomitant increase in Sp suppressor gene expression. The betulinic acid analogs and derivatives also are effective in methods provided herein for inhibiting proliferation of cells associated with a neoplastic disease for treating a cancer or for reducing toxicity of a cancer therapy in a subject via administration of an analog or derivative of betulinic acid and, optionally, another anticancer drug.

### CROSS-REFERENCE TO RELATED APPLICATION

This nonprovisional application claims benefit of provisional U.S. Ser. No. 60/851,113, filed Oct. 12, 2006, now abandoned.

### FEDERAL FUNDING LEGEND

This invention was produced in part using funds obtained through the National Institutes of Health Grant Nos. ES09106, CA108718 and CA112337. Consequently, the federal government has certain rights in this invention.

### BACKGROUND OF THE INVENTION

1. Field of the Invention

The present invention relates generally to the fields of organic chemistry and cancer treatment. More specifically, the present invention relates to betulinic acid, derivatives and analogs thereof and their uses as agents against cell proliferative diseases.

2. Description of the Related Art

Phytochemicals have been used extensively in therapeutic applications to treat multiple diseases including cancer chemotherapy and prevention. Among the diverse structural classes of natural products used for cancer chemotherapy, triterpenoids are highly promising models for drug development. Betulinic acid (BA) is a triterpenoid acid natural product and is readily synthesized from betulin (lup-20(29)-ene-3β,28-diol), a major constituent of birch bark. Initial studies showed that it was a highly selective inhibitor of human melanoma cell proliferation through induction of apoptosis [1].

The development of synthetic derivatives of natural phytochemical products is an on going process. For example, U.S. Pat. Nos. 5,679,828, 5,869,535 and 6,048,847 disclose synthetic derivatives of betulinic acid and cytotoxicity studies against disease-associated and normal cells. However, although newer high throughput screening techniques and the development of chemical libraries has facilitated discovery of many new synthetic drugs, the overall percentage of new drugs derived from natural products or their synthetic analogs approximately 25%. Therefore, a continuing need for new chemotherapeutics derived from natural phytochemical products and improved methods of cancer therapy is present in the art.

Thus, the prior art is still deficient in the lack of betulinic acid derivatives and analogs that are effective as chemotherapeutics. More specifically, the prior art is deficient in the lack of betulinic acid and its derivatives and analogs that are useful in and primarily act by degrading Sp proteins and/or as modulators of peroxisome proliferator-activated receptor γ (PPARγ) agonist responses in neoplastic cells. The present invention fulfills this long-standing need and desire in the art.

### SUMMARY OF THE INVENTION

The present invention is directed to a betulinic acid analog or derivative compound having the structural formula:

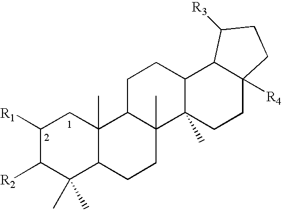

where a bond between C1 and C2 is a single bond or a double bond, R₁ is H, CN, Cl, Br, F, I, CH₃, CF₃, OCH₃, N(CH₃)₂, or phenyl, R₂ is OH or ═O, R₃ is COOH, COOCH₃, COOCH₂CH₃, or CHO, and R₄ is CH(CH₃)₂ or C(CH3)═CH₂ or a pharmacologically effective salt or hydrate thereof.

The present invention is direct to a related compound that is a synthetic analog or derivative of betulinic acid having the structural formula described herein where R₁ is CN, CH₃, CF₃, OCH₃, N(CH₃)₂, or phenyl, R₂ is OH or ═O, R₃ is COOH, COOCH₃, COOCH₂CH₃, or CHO, and R₄ is CH(CH₃)₂ or C(CH3)=CH₂ or a pharmacologically effective salt or hydrate thereof. The present invention is directed to a related betulinic acid analog or derivative where R₁ is CN, R₂ is ═O and R₃ is COOH or COOCH₃. The present invention is directed to another related betulinic acid analog or derivative where R₃ is COOCH₂CH₃ or CHO and R₁ further comprises Cl, Br, F, or I.

The present invention also is directed to a method for inhibiting an activity of one or more specificity protein (Sp) transcription factors in a cell associated with a neoplastic disease. The method comprises decreasing expression of a microRNA in the cell via contact with one or more of betulinic acid or the betulinic acid analog or derivative compounds described herein or a combination thereof thereby increasing expression of an Sp repressor gene in the cell.

The present invention is directed further to a method for inhibiting the proliferation of cells associated with a neoplastic disease. The method comprises contacting the cell with betulinic acid, a betulinic acid analog or derivative described herein or a combination thereof. The present invention is directed to a related method where the cells form a tumor associated with the neoplastic disease in a subject and comprises a further method step of administering a pharmacologically effective amount of one or more other anticancer drugs to the subject.

The present invention is directed further still to a method for treating a cancer in a subject. The method comprises administering a pharmacologically effective amount of one or more of the betulinic acid analog or derivative compounds described herein to the subject where the compound inhibits growth of cancer cells thereby treating the cancer.

The present invention is directed further still to a method of reducing toxicity of a cancer therapy in a subject in need thereof. The method comprises administering to the subject a pharmacologically effective amount of one or more of the betulinic acid analog or derivative compounds described herein and another anticancer drug. A dosage of the anticancer drug administered with the betulinic acid analog(s) and/or derivative(s) thereof is lower than a dosage required when the anticancer drug is administered singly, thereby reducing toxicity of the cancer therapy to the individual.

Other and further aspects, features and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1D demonstrate that betulinic acid inhibits growth and induces apoptosis in cancer cells. Decreased cell survival in LNCaP prostate cancer cells (FIG. 1A) and SK-MEL2 melanoma cells (FIG. 1B). Cells were seeded, treated with solvent (DMSO) or different concentrations of betulinic acid (1-20 μM) for 6 days as described. Cell survival is expressed as the percentage of betulinic acid-treated cells remaining compared to DMSO (set at 100%), and significantly (p<0.05) decreased survival is indicated by an asterisk. FIGS. 1C-1D are Western blot analyses for modulation of protein expression by betulinic acid. LNCaP cells were treated with DMSO or betulinic acid (5-20 μM) for 24 hr and whole cell lysates were analyzed.

FIGS. 2A-2D demonstrate that betulinic acid induces degradation of Sp and other proteins in LNCaP cells. Decreased expression of Sp1, Sp3, Sp4 and VEGF in LNCaP (FIGS. 2A-2C) and SK-MEL2 (FIG. 2D) cells is shown. LNCaP or SK-MEL2 cells were treated with DMSO or BA (1-20 μM) for 24 (FIGS. 2A and 2D), 4-24 (FIG. 2B), 48 or 72 (FIG. 2C) hr, and whole cell lysates were analyzed by Western blot analysis.

FIGS. 3A-3D demonstrate that betulinic acid induces proteasome-dependent degradation of Sp proteins in LNCaP cells. FIG. 3A shows the effects of cycloheximide. Cells were cotreated with 10-20 μM betulinic acid or 10 μg/ml cycloheximide alone or in combination, and expression of Sp proteins in whole cell lysates was determined by immunoblot analysis. FIGS. 3B-3D show the effects of the proteasome inhibitor MG132 on betulinic acid induced decrease of Sp proteins/VEGF (FIG. 3B), PARP cleavage, cyclin D1 and AR (FIG. 3C), and PARP cleavage and survivin (FIG. 3D). LNCaP cells were treated with DMSO or betulinic acid alone or in combination with 5 or 10 μM MG132 (pretreated for 30 min) for 24 hr, and protein expression in whole cell lysates was analyzed by Western blot.

FIGS. 4A-4D demonstrate that betulinic acid decreases transactivation in LNCaP cells transfected with VEGF and survivin constructs. Transfection with pVEGF-2068 (FIG. 4A), pVEGF-133 (FIG. 4B), pSurvivin-269 (FIG. 4C), and pSurvivin-150 (FIG. 4D) is shown. LNCaP cells were transfected with the various constructs, treated with DMSO or betulinic acid (2.5-20 μM) alone or in combination with 10 μM MG132, and luciferase activity (relative to β-gal) was determined. Luciferase activity significantly (p<0.05) decreased by betulinic acid (*) and inhibition of this response by cotreatment with MG132 (**) is indicated.

FIGS. 5A-5B demonstrate that VEGFR1 expression is Sp-dependent. In cells transfected with siRNA (iSp1, iSp2 or iSp3), degradation of Sp proteins also caused a decrease in VEGFR1 protein (FIG. 5A) and in cells transfected with the siRNAs and VEGFR1 promoter construct a decrease in luciferase activity was present (FIG. 5B) in pancreatic cancer Panc-1 cells.

FIGS. 6A-6F demonstrate antitumorigenic activity by betulinic acid in vivo. FIG. 6A shows a decreased tumor area. Athymic nude mice (10 per group) bearing LNCaP cells as xenografts were treated with corn oil (control) or BA in corn oil (10 or 20 ml/kg) every second day and tumor areas were determined. FIG. 6B shows tumor weights. After the final treatment, animals were sacrificed and tumor weights were determined. Significantly (p<0.05) decreased tumor areas or volumes are indicated by an asterisk. FIGS. 6C-6F are histopathological evaluation of tumors. Tumors from corn oil (FIGS. 6C-6D) and betulinic acid (FIGS. 6E-6F) mice were fixed, stained with hematoxylin and eosin and examined histopathologically.

FIGS. 7A-7D demonstrate Sp and VEGF protein expression in tumors and liver. Whole cell lysates from corn oil (untreated) and BA-treated tumors (FIG. 7A) and liver (FIG. 7B) were obtained from tissue from at least 5 rats per group and analyzed by Western blot analysis. FIG. 7C shows the comparative Sp protein expression in tumors and liver from corn oil (solvent)-treated animals. Tumor and liver lysates containing the same amount of protein were analyzed by electrophoresis and visualized. FIG. 7D shows immunostaining for CD31 and VEGF. Fixed tumor tissue from corn oil and BA-treated mice were stained with CD31 and VEGF antibodies.

FIGS. 8A-8I demonstrate cell proliferation and adipocyte differentiation assays. Panc-28 and SW480 cells were treated with different concentrations of betulinic acid (FIGS. 8A-8B), CN-BA (FIGS. 8C-8D) or CN-BA-Me (FIGS. 8E-8F) for 6 days and the number of cells were counted after treatment for 2, 4 or 6 days. Results are expressed as means±SE for three separate determinations for each treatment group. FIGS. 8G-2I show effects of CN-BA and CN-BA-Me on differentiation of 3T3-L1 adipocytes. 3T3-L1 adipocytes were treated with 0.25 μM CN-BA, CN-BA-Me or DMSO. Induction of fat droplets by Oil-red O staining was determined. Induction of intense staining for fat droplets was observed in replicate (3) experiments.

FIGS. 9A-9G demonstrate activation of PPARγ in SW480 and Panc-28 cells by betulinic acid, CN-BA and CN-BA-Me. SW480 cells were transfected with PPARγ-GAL4/pGAL4 (FIGS. 9A & 9C) or PPRE₃-luc (FIGS. 9B & 9D) treated with DMSO (control) or different concentrations of the compounds, and luciferase activity was determined. FIGS. 9E-9F show activation of PPARγ in Panc-28 cells. Cells were transfected with PPARγ-GAL4/pGAL4 (FIG. 9E) or PPRE₃-luc (FIG. 9F) treated with DMSO or different concentrations of CN-BA and CN-BA-Me alone or in combination with 10 μM T007, and luciferase activity determined as described. Results in FIGS. 9A-9F are expressed as means±SE for three replicate determinations for each treatment group, and significant (p<0.05) induction by the BA derivatives (*) and inhibition after cotreatment with T007 (**) are indicated. FIG. 9G shows a mammalian two-hybrid assay in SW480 cells transfected with VP-PPARγ and GAL4-coactivator chimeras. SW480 cells were transfected with VP-PPARγ, coactivator-GAL4/pGAL4, treated with different concentrations of CN-BA or CN-BA-Me and 5 μM β-CDODA-Me, and luciferase activity was determined. Results are expressed as means±SE for three replicate determinations for each treatment group, and significant (p<0.05) induction is indicated by an asterisk.

FIGS. 10A-10F demonstrate induction of p21 by BA, CN-BA and CN-BA-Me in Panc-28 cells. FIG. 10A shows the induction of p21 protein. Panc-28 cells were treated with the different compounds as indicated for 24 hr and whole cell lysates were obtained and analyzed by immunoblots. Induction of p21-luc (FIG. 10B) and p21 deletion constructs (FIG. 10C) in Panc-28 cells is shown. Cells were transfected with the various constructs, treated with DMSO, BA, CN-BA, CN-BA-Me alone or in combination with T007, and luciferase activity determined. Results of all transactivation studies in this Figure are presented as means±SE for at least three separate determinations for each treatment group. Significant (p<0.05) induction compared to solvent (DMSO) control (*) and inhibition by cotreatment with T007 (**) are indicated. FIGS. 10D-10F show chromatin immunoprecipitation assays. Primers designed for the proximal region of the p21 promoter (FIG. 10D) were used for a ChIP assay in Panc-28 cells (FIG. 10E) treated with DMSO, 5 μM BA, 5 μM CN-BA, and 5 μM CN-BA-Me for 1 or 2 hr. Analysis of interactions of Sp1 and PPARγ with the p21 promoter were carried out in the ChIP assay as described. The ChIP assay was also used to examine binding of TFIIB to the GAPDH promoter (positive control) (FIG. 10F) and to exon 1 of CNAP1 (negative control).

FIGS. 11A-11E demonstrate the induction of caveolin-1 expression in colon cancer cells. HT-29 (FIG. 11A), HCT-15 (FIG. 11B) and SW480 (FIG. 11C) cells were treated with DMSO, different concentrations of BA, CN-BA or CN-BA-Me for 72 hr. Caveolin-1 expression was determined by Western blot analysis. Similar results were observed in replicate experiments. FIGS. 11D-11E show the effects of T007 on induction of caveolin-1. HCT-29 (FIG. 11D) or HT-15 (FIG. 11E) cells were treated with DMSO or different concentrations of CN-BA and CN-BA-Me alone or in combination with 5 μM T007 and caveolin-1 expression was determined by Western blot analysis.

FIGS. 12A-12D demonstrate induction of KLF4 gene expression apoptosis by BA and related compounds. Induction of KLF4 in HT-29 (FIG. 12A) and SW480 (FIG. 12B) cells. Cells were treated with different concentrations of CDODA isomers or T007 alone or in combination, and KLF4 mRNA levels were determined by real time PCR. Each experiment was replicated (>3×) and T007 did not inhibit KLF4 mRNA induction by betulinic acid, CN-BA and CN-BA-Me, whereas 60-80% of the response induced by β-CDODA-Me was inhibited by T007. KLF4 mRNA levels were not induced in SW480 cells by BA derivatives. FIGS. 12C-12D show induction of apoptosis. Panc28 (FIG. 12C) and SW480 (FIG. 12D) cells were treated for 24 hr with betulinic acid and related compounds and whole cell lysates were examined by Western blot analysis.

FIGS. 13A-13C demonstrate in vivo that CN-BA inhibits cell proliferation in LNCaP prostate cancer cells (FIG. 13A) and Ku-7 bladder cancer cells (FIG. 13B) and activates a PPARγ-GAL4 chimera (FIG. 13C).

FIG. 14 demonstrates that BA and CN-BA activate proapoptotic responses in RKO cells. RKO cells were transfected with BA or CN-BA for 24 hr and whole cell lysates were examined by Western blot analysis.

FIGS. 15A-15C demonstrate that BA and/or BA-CN decrease Sp protein expression in colon and pancreatic cancer cells. RKO colon cancer cells were treated with BA/CN-BA for 24 hr (FIG. 15A) or 48 hr (FIG. 15B) and Miapaca-2 pancreatic cancer cells were treated with BA/CN-BA for 24 hr (FIG. 15C). Whole cell lysates were examined by Western blot analysis.

FIGS. 16A-16G demonstrate the effects of BA, CN-BA and/or CN-BA-M3 on expression of miR-27a in colon and pancreatic cancer cells. FIG. 16A shows expression of miR-27a in RKO, SW480, HT-29, Panc-28, and LNCaP cells. FIG. 16B shows the effects of BA on miR-27a in RKO colon cancer cells. FIG. 16C shows the effects of BA on miR-27a Panc-28 pancreatic cancer cells. FIG. 16D shows the effects of BA, CN-BA and CN-BA-Me on ZBTB10 mRNA levels in HT-29 colon cancer cells. FIG. 16E shows the effects of BA and CN-BA on ZBTB10 mRNA levels in Miapaca-2 pancreatic cancer cells. FIG. 16F shows the effects of ZBTB10 overexpression in SW480 cells. FIG. 16G shows the effects of ZBTB10 overexpression in Miapaca-2 pancreatic cancer cells. Cells were transfected with varying amounts of ZBTB10. Whole cell lysates were examined by Western blot analysis.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, the term “contacting” refers to any suitable method of bringing betulinic acid, its derivatives and analogs or any other therapeutic compound into contact with a cell, preferably with an abnormally proliferating cell. In vitro or ex vivo this is achieved by exposing the cells to the inhibitory agent in a suitable medium. For in vivo applications, any known method of administration is suitable.

As used herein, the term “treating” or the phrase “treating a cancer” includes, but is not limited to, halting the growth of cancer cells, killing the cancer cells or a mass comprising the same, or reducing the number of cancer cells or the size of a mass comprising the same. Halting the growth refers to halting any increase in the size or the number of cancer cells or in a mass comprising the same or to halting the division of the cancer cells. Reducing the size refers to reducing the size of a mass comprising the cancer cells or the number of or size of the same cells. As would be apparent to one of ordinary skill in the art, the term “cancer” or “cancer cells” or “tumor” refers to examples of neoplastic cell proliferative diseases and refers to a mass of malignant neoplastic cells or a malignant tissue comprising the same.

As used herein, the term “degrading” or “degradation” of Sp transcription factors in cells associated with a neoplastic disease, e.g., cells comprising a cancer or tumor or malignant or abnormally proliferating cells, shall include partial or total degradation of one or more Sp proteins and also is meant to include decreases in the rate of proliferation or growth of the cells associated with the neoplastic disease. The biologically, pharmacologically or therapeutically effective dose of the compounds of the present invention may be determined by assessing the effects of the compounds on Sp protein degradation in target malignant or abnormally proliferating cells in tissue culture or cell culture, on tumor growth in animals or any other method known to those of ordinary skill in the art.

As used herein, the term “subject” refers to any target of the treatment.

In one embodiment of the present invention there is provided a betulinic acid analog or derivative having a structural formula:

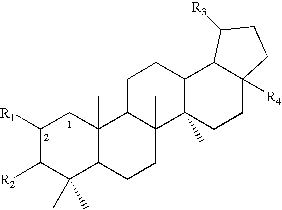

wherein a bond between C1 and C2 is a single bond or a double bond, R₁ is H, CN, Cl, Br, F, I, CH₃, CF₃, OCH₃, N(CH₃)₂, or phenyl; R₂ is OH or ═O; R₃ is COOH, COOCH₃, COOCH₂CH₃, or CHO; and R₄ is CH₃ and R⁵ is H or R₄ is CH(CH₃)₂ or C(CH3)=CH₂; or a pharmacologically effective salt or hydrate thereof. In one aspect R₁ may be Cl, Br or CN. In another aspect R₂ may be ═O. In yet another aspect R₃ may be COOH or COOCH₃. In yet another aspect C1-C2 is a double bond, R₁ may be CN, R₂ may be ═O and R₃ may be COOH or COOCH₃. In yet another aspect R₁ may be Cl or Br, R₂ may be ═O, and R₃ may be COOCH₂CH₃ or CHO.

In a related embodiment there is provided a synthetic analog or derivative of betulinic acid having the general structural formula as described supra. In this embodiment a bond between C1 and C2 is a single bond or a double bond, the structural substituent R₁ is CN, CH₃, CF₃, OCH₃, N(CH₃)₂, or phenyl, R₂ is OH or ═O, R₃ is COOH, COOCH₃, COOCH₂CH₃, or CHO, and R₄ is CH(CH₃)₂ or C(CH3)=CH₂ or comprises a pharmacologically effective salt or hydrate thereof. In this embodiment R₁ may be CN, R₂ may be ═O and R₃ may be COOH or COOCH₃. Also, the substituent R₃ may be COOCH₂CH₃ or CHO where R₁ further comprises Cl, Br, F, or I.

In another embodiment of the present invention there is provided a method for inhibiting an activity of one or more specificity protein (Sp) transcription factors in a cell associated with a neoplastic disease, comprising decreasing expression of a microRNA in the cell via contact with one or more of betulinic acid or the betulinic acid analog or derivative compounds described supra or a combination thereof thereby increasing expression of an Sp repressor gene in the cell.

In this embodiment the Sp transcription factor may be an Sp1 protein, an Sp3 protein, an Sp4 protein or a combination thereof. Also, the microRNA may be miR-27a. In addition the Sp suppressor gene may be ZBTB10. Furthermore, the neoplastic disease may be a kidney cancer, a bladder cancer or prostate cancer, a colon cancer, a melanoma, a pancreatic cancer, or a breast cancer.

In yet another embodiment of the present invention there is provided a method for inhibiting proliferation of cells associated with a neoplastic disease, comprising contacting the cell with one or more of betulinic acid or the betulinic acid analog or derivative compounds described supra or a combination thereof. Further to this embodiment, the cells may form a tumor associated with the neoplastic disease in a subject where the method comprises administering a pharmacologically effective amount of one or more other anticancer drugs to the subject.

In both embodiments, betulinic acid or the analog or derivative compounds thereof may be effective to increase expression of Sp suppressor gene ZBTB10, induce degradation of one or more Sp transcription factors in the cells, or to activate PPARγ responses in the cells or a combination thereof. The Sp transcription factor may be an Sp1 protein, an Sp3 protein, an Sp4 protein or a combination thereof. The neoplastic disease is as described supra.

In another embodiment of the present invention there is provided method for treating a cancer in a subject, comprising administering a pharmacologically effective amount of one or more of the betulinic acid analog or betulinic derivative compounds described supra to the subject, where the compound inhibits growth of cancer cells thereby treating the cancer.

In this embodiment the betulinic acid analog or derivative compound(s) may be effective to inhibit angiogenesis in a tumor associated with the cancer and/or may be effective to inhibit metastasis of cancer cells associated with the cancer. The specific cancers may be the neoplastic diseases as described supra.

In yet another embodiment of the present invention, there is provided a method for reducing toxicity of a cancer therapy in a subject in need thereof, comprising administering to the subject a pharmacologically effective amount of one or more of a betulinic acid analog or derivative compound described supra and another anticancer drug, such that a dosage of the anticancer drug administered with the betulinic acid analog or derivative compound(s) is lower than a dosage required when the anticancer drug is administered singly, thereby reducing toxicity of the cancer therapy to the individual. In this embodiment the anticancer drug may be administered concurrently or sequentially with the betulinic acid analog or derivative. Also, the cancers are described supra.

The present invention provides betulinic acid and its derivatives and analogs, or a pharmacologically acceptable salt or hydrate thereof, that exhibit a therapeutic effect on cells associated with a neoplastic or cell proliferative disease in vitro and in vivo. These betulinic acid compounds induce Sp protein degradation e.g., Sp1, Sp3 and Sp4, in a proteosome-independent manner. It is contemplated that this effect is linked to modulation of microRNA, e.g., miR27-a expression which, in turn, regulates the Sp-repressor ZBTB10. Also, as an agonist, the compounds provided herein modulate PPARγ responses. In addition, these compounds induce receptor-independent growth inhibitory and/or proapoptotic responses such as, but not limited to, activation of the JNK pathway, ATF-3 and NAG-1.

These compounds are synthesized using well-known and standard techniques in the chemical synthetic arts (2-3). Generally, these compounds may be, although not limited to, betulinic acid, dihydrobetulinic acid, the keto analogs betulonic acid and dihydrobetulonic acid, and derivatives thereof substituted at, although not limited to, one or more of C2 or C28. In preferable non-limiting examples, a 2-cyano group is introduced into the lupane skeleton of 20(29)-dihydro betulinic acid or the corresponding methyl ester is used.

Numbering of the carbon atoms and rings in these compound structures is based on betulinic acid and uses standard protocol. The numbering protocol is continued with any betulinic acid analog compound, e.g., betulonic acid and its derivatives. Thus, the C3 substitutent in the A ring determines if the compound is a betulinic acid or the analog betulonic acid and the C20 substituents in the E ring determine if the compound is the dihydro analog. Also, a double bond between C1 and C2 forms an ene analog or derivative thereof.

More particularly, a compound effective is encompassed by, but not limited to, the structural formula:

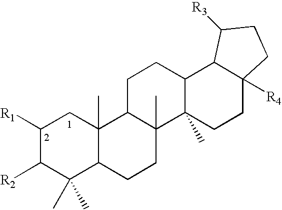

In this structure a single or a double bond may be formed between carbons C1 and C2. R₁ may be hydrogen, a cyano group, a halide, i.e., chlorine, bromine, fluorine or iodine, an alkyl group, e.g., methyl, a haloalkyl group, e.g., trifluoromethyl, an alkylamine, e.g., dimethyl amine, an alkoxy group, e.g., methoxy, or a phenyl group. R₂ may be hydroxy or a carbonyl oxygen. R₃ may be a carboxy group, an alkyl ester, e.g. a C1-C4 alkyl ester, preferably methyl ester or ethyl ester, an aldehyde, e.g., formaldehyde, or an amide or alkyl substituted amide, which may be further substituted, R₄ may form the dihydro isopropyl moiety CH(CH₃)₂ or may form the methylethylene moiety C(CH₃)═CH₂, as in betulinic acid or betulonic acid. Preferably, R₁ is a cyano group, a chlorine or a bromine. Also, in another preferable embodiment R₂ is a carbonyl oxygen. In yet another preferable embodiment, R₃ is a carboxy group or the ester, particularly, the methyl or ethyl ester, thereof. Alkyl substituents may be straight- or branched-chain or cycloalkyl or, for longer chains may be the alkenyl or alkynyl derivative.

Thus, the betulinic acid and its derivatives and analogs provided herein are useful as therapeutics or chemotherapeutics to inhibit growth of abnormally proliferating cells in a neoplastic or cell proliferative disease. It is contemplated that contacting the abnormally proliferating or neoplastic cells with one or more of these compounds is effective to induce at least apoptosis in the cells. Therefore, the compounds of the present invention are useful in treating cancers in a subject. It also is contemplated that the therapeutic effect would result in inhibition of metastasis of the cancer cells and/or inhibit angiogenesis. Preferably the subject is a mammal, more preferably the subject is a human. Examples of cancers are, although not limited to, genitourinary cancers, such as a bladder cancer, a kidney cancer or prostate cancer, gastrointestinal cancers, pancreatic cancers, melanomas, or breast cancers.

Furthermore, an anticancer drug may be administered concurrently or sequentially with betulinic acid or its analogs and derivatives. Betulinic acid, analogs and derivatives thereof provided herein and other anticancer drugs can be administered independently, either systemically or locally, by any method standard in the art, for example, subcutaneously, intravenously, parenterally, intraperitoneally, intradermally, intramuscularly, topically, enterally, rectally, nasally, buccally, vaginally or by inhalation spray, by drug pump or contained within transdermal patch or an implant.

The effect of co-administration is to lower the dosage of the anticancer drug normally required that is known to have at least a minimal pharmacological or therapeutic effect against a genitourinary cancer or cancer cell associated therewith, for example, the dosage required to eliminate a cancer cell. Concomitantly, toxicity of the anticancer drug to normal cells, tissues and organs is reduced without reducing, ameliorating, eliminating or otherwise interfering with any cytotoxic, cytostatic, apoptotic or other killing or inhibitory therapeutic effect of the drug on the cancer cells. Examples of anticancer drugs effective to treat a cancer, particularly the cancers described herein, are known in the art.

Dosage formulations of these betulinic acid derivative and analog compounds or a pharmacologically acceptable salt or hydrate thereof or other anticancer drug may comprise conventional non-toxic, physiologically or pharmaceutically acceptable carriers or vehicles suitable for the method of administration. These compounds or pharmaceutical compositions thereof may be administered independently one or more times to achieve, maintain or improve upon a pharmacologic or therapeutic effect derived from these compounds or other anticancer drugs or agents. It is well within the skill of an artisan to determine dosage or whether a suitable dosage comprises a single administered dose or multiple administered doses. An appropriate dosage depends on the subject's health, the progression or remission of the cancer, the route of administration and the formulation used.

The following example(s) are given for the purpose of illustrating various embodiments of the invention and are not meant to limit the present invention in any fashion.

### Example 1

### Synthesis of Betulinic Analogs and Derivatives

2-chloro and 2-bromo Derivatives of Dihydrobetulonic Acid

As previously described [2], dihydrobetulonic acid methyl ester is treated with phenylselenyl chloride and the m-chloroperbenzoic acid (mCIBPA) to form the 1-ene derivative at step 1. This is converted at step 2 into the 1,2-epoxy derivative by the treatment with hydrogen peroxide. Subsequent treatment of the epoxide with HCl or HBr at step 3 followed by deesterification with LiI at step 4 yields 2-chlorodihydro- or 2-bromodihydro-betulonic

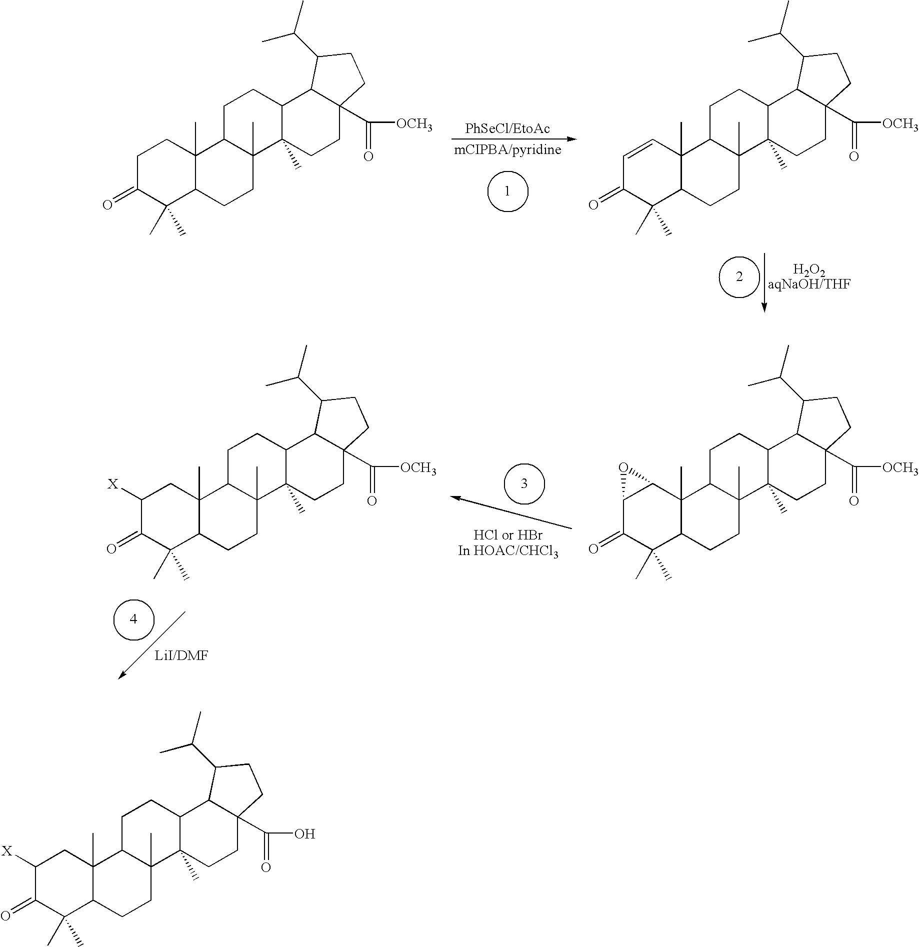

acid where X is chloro or bromo.

### Synthesis of CN-BA and CN-BA-Me

CN-BA and CN-BA-Me were prepared from betulin (Sigma-Aldrich) based on the previous methods [2]. The synthesis from a key intermediate, methyl lup-2-eno[2,3-d]isoxazol-28-oate, is briefly described and only definite peaks in proton NMR are recorded.

Methyl lup-2-eno[2,3-d]isoxazol-28-oate

To a solution of methyl lupan-2-hydroxymethylene-3-oxo-28-oate (350 mg, 0.70 mmoL) in ethanol (20 mL) and water (1 mL), hydroxylamine hydrochloride (488 mg, 7.02 mmol) was added. The reaction mixture was refluxed for 1 hr, cooled to room temperature and concentrated under vacuum. Water was added to the reaction mixture and extracted with ethyl acetate (2×). The organic layer was then washed with brine (2×), separated, and the crude product was purified by a flash silica gel column using a solvent system of hexanes:ethyl acetate (95:5) to yield methyl lup-2-eno[2,3-d]isoxazol-28-oate as a white cream colored solid. ¹H NMR (400 MHz, CDCl₃): δ 8.00 (1H, s), 3.68 (3H, s), 1.31, 1.21, 0.99, 0.98, 0.83 (each 3H, s), 0.89 (3H, d, J=6.8 Hz), 0.78 (3H, d, J=6.8 Hz). ¹³C NMR (100 MHz, CDCl₃): δ 177.6, 173.8, 151.1, 109.7, 57.8, 54.3, 52.0, 49.7, 49.6, 45.0, 43.4, 41.5, 39.7, 38.9, 38.1, 36.6, 35.6, 34.2, 32.8, 30.6, 30.5, 30.4, 29.5, 27.7, 23.8, 23.6, 22.2, 22.0, 19.6, 16.8, 16.5, 15.5, 15.4.

Methyl 2-cyano-lup-3-hydroxy-2-en-28-oate

To a solution of methyl lup-2-eno[2,3-d]isoxazol-28-oate (250 mg, 0.50 mmol) in ether (30 mL) and methanol (15 mL) in an ice bath, 30% sodium methoxide in methanol (3071 mg, 56.87 mmol) was added drop wise. The reaction mixture was then stirred at room temperature for 2 hr. After dilution with ether, the reaction mixture was washed with 5% hydrochloric acid (2×). The organic layer was separated and worked up by standard method to yield crude methyl 2-cyano-lup-3-hydroxy-2-en-28-oate (240 mg, 96%) as a white solid, which was used for the next step without further purification. ¹H NMR (400 MHz, CDCl₃): δ 3.90 (1H, m), 3.67 (3H, s), 1.27, 1.17, 0.98, 0.95, 0.83 (each 3H, s), 0.89 (3H, d, J=6.8 Hz), 0.79 (3H, d, J=6.8 Hz).

Methyl 2-cyano-lup-1-en-3-oxo-20-oate (CN-BA-Me)

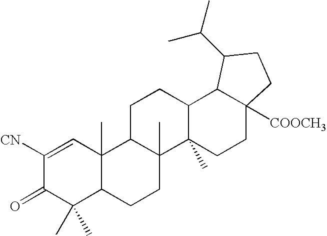

A mixture of methyl 2-cyano-lup-3-hydroxy-2-en-28-oate (230 mg, 0.46 mmol) and DDQ (117 mg, 0.52 mmol) in benzene (30 mL) was refluxed for 3 hr. The reaction mixture was cooled in ice and filtered to remove reduced DDQ. The filtrate was then concentrated under vacuum. The crude product was purified by a flash silica gel column using a solvent system of benzene:acetone (98:2) to yield CN-BA-Me (170 mg, 74%) as a pale brown solid. ¹H NMR (400 MHz, CDCl₃): δ 7.83 (1H, s), 3.66 (3H, s), 1.25, 1.18, 1.12, 1.00, 0.95 (each 3H, s), 0.87 (3H, d, J=6.7 Hz), 0.75 (3H, d, J=6.6 Hz). ¹³C NMR (100 MHz, CDCl₃): δ 199.0, 177.4, 171.6, 115.8, 114.6, 57.6, 53.3, 52.0, 49.4, 45.7, 44.8, 44.4, 43.6, 42.6, 41.5, 38.8, 37.9, 34.2, 32.6, 30.4, 30.2, 28.5, 27.3, 23.7, 23.5, 22.1, 22.0, 19.6, 19.1, 17.2, 15.4, 15.2. ESI-HRMS Calcd for (C₃₂H₄₇NO₃+H): 494.3634. Found: 494.3685. Anal. (C₃₂H₄₇NO₃) C, H.

2-Cyano-lup-1-en-3-oxo-20-oic acid (CN-BA)

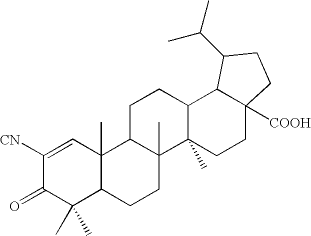

A mixture of CN-BA-Me (120 mg, 0.25 mmol) and lithiumiodide (720 mg) in dimethylformamide (2.4 mL) was refluxed for 2 hr. The reaction mixture was cooled to room temperature and 5% hydrochloric acid was added. The reaction mixture was extracted with ethyl acetate (2×). The organic layer was then washed with water (2×) followed by washings with brine (2×). The organic layer was separated and worked up by standard method. The crude product was purified by a flash silica gel column using a solvent system of hexanes:ethyl acetate (80:20) to yield CN-BA (93 mg, 80%) as a light yellow solid. ¹H NMR (400 MHz, CDCl₃): δ 10.29 (1H, broad s), 7.83 (1H, s), 1.27, 1.14, 1.04, 0.99, 0.99 (each 3H, s), 0.91 (3H, d, J=6.8 Hz), 0.79 (3H, d, J=6.7 Hz). ¹³C NMR (100 MHz, CDCl₃): δ 199.0, 182.1, 171.5, 115.8, 114.8, 57.5, 53.3, 49.3, 45.8, 44.8, 44.4, 43.7, 42.7, 41.5, 39.0, 38.1, 34.3, 32.7, 32.7, 30.5, 30.3, 28.6, 27.4, 23.8, 23.5, 22.2, 22.1, 19.6, 19.2, 17.3, 15.4, 15.3. ESI-HRMS Calcd for (C₃₁H₄₅NO₃+H): 480.3478. Found: 480.3540. Anal. (C₃₁H₄₅NO₃) C, H. CN-BA and CN-BA-Me were >97% pure by spectroscopic analysis.

Structure of Methyl 2-cyano-3,12-dioxo-18β-olean-1,12-diene-30-oic Acid (β-CDODA-Me)

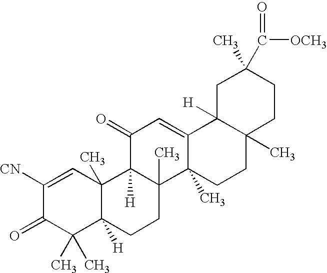

### Example 2

### In Vitro Effects of Betulinic Acid on Genitourinary Cancer Cells

Betulinic acid and beta-actin antibody were purchased from Sigma Aldrich (St. Louis, Mo.) and proteasome inhibitor MG132 was purchased from Calbiochem (San Diego, Calif.). Antibodies against Sp1, Sp4, Sp3, VEGF, CD1, AR, KLF6, survivin and PARP were obtained from Santa Cruz Biotechnology (Santa Cruz, Calif.) and CD31 antibody from DakoCytomation (Glostrup, Denmark). The pVEGF-2018 and pVEGF-133 constructs contain VEGF promoter inserts (positions −2018 to +50 and positions −131 to +54, respectively) linked to luciferase reporter gene [4]. The pSurvivin-269 and pSurvivin-150 were provided by Dr. M. Zhou (Emory University, Atlanta, Ga.). Reporter lysis buffer and luciferase reagent for luciferase studies were purchased from Promega (Madison, Wis.). Beta-galactosidase (β-gal) reagent was obtained from Tropix (Bedford, Mass.). Lipofectamine reagent was supplied by Invitrogen (Carlsbad, Calif.). Western Lightning chemiluminescence reagent was from Perkin-Elmer Life Sciences (Boston, Mass.).

Human carcinoma cell lines LNCaP (prostate) and SK-MEL2 (melanoma) were obtained from American Type Culture Collection (Manassas, Va.). Cell lines were maintained in RPMI 1640 (Sigma) supplemented with 0.22% sodium bicarbonate, 0.011% sodium pyruvate, 0.45% glucose, 0.24% HEPES, 10% FBS, and 10 mL/L of 100× Antibiotic Antimycotic solution (Sigma). Cells were maintained at 37° C. in the presence of 5% CO2.

Prostate and melanoma cancer cells (2×104 per well) were plated in 12-well plates and allowed to attach for 24 hr. The medium was then changed to DMEM:Ham's F-12 medium containing 2.5% charcoal-stripped FBS and either vehicle (DMSO) or different concentrations of the compound were added. Fresh medium and compounds were added every 48 hr and cells were then trypsinized and counted after 48 and 96 hr using a Coulter Z1 cell counter. Each experiment was done in triplicate and results are expressed as means±SE for each set of experiments.

Prostate cancer cells were plated in 12-well plates at 1×10⁵ cells/well in DMEM:Ham's F-12 media supplemented with 2.5% charcoal-stripped FBS. After growth for 16-20 hr, various amounts of reporter gene constructs, i.e., pVEGF-2018 (0.4 μg), pVEGF-133 (0.04 μg), pSurvivin-269 (0.04 μg), pSurvivin-150 (0.04 μg) and β-gal (0.04 μg), were transfected by Lipofectamine (Invitrogen) according to the manufacturer's protocol. After 5 hr of transfection, the transfection mix was replaced with complete media containing either vehicle (DMSO) or the indicated compound for 20 to 22 hr. Cells were then lysed with 100 μL of 1× reporter lysis buffer and 30 μL of cell extract were used for luciferase and β-gal assays. Lumicount was used to quantitate luciferase and β-gal activities and the luciferase activities were normalized to β-gal activity.

An equal amount of cell lysate (60 μg/well) was separated by 7.5% to 12% SDS-PAGE, which was followed by immunoblotting onto polyvinylidene difluoride (Bio-Rad, Hercules, Calif.). After blocking in TBST-Blotto (10 mmol/L Tris-HCl, 150 mmol/L NaCl (pH 8.0), 0.05% Triton X-100, 5% nonfat dry milk) for 30 min, the membranes were incubated with primary antibodies overnight at 4° C. and then with horseradish peroxidase-conjugated secondary antibody for 2 hr at room temperature. Proteins were visualized using the chemiluminescence substrate (Perkin-Elmer Life Sciences) for 1 min and exposed to Kodak X-OMAT AR autoradiography film (Eastman Kodak, Rochester, N.Y.).

Male athymic BALB/c nude mice (age 4-6 weeks) were purchased from Harlan (Indianapolis, Ind.). LNCaP cells (1×106) were implanted with matrigel (BD Biosciences, San Jose, Calif.) subcutaneously into the flank of each mouse. Ten days after cell inoculation, animals were divided into three groups of 10 mice each. The first group received 100 μL vehicle (1% DMSO in corn oil) by oral gavage and the second and third groups of animals received 10 and 20 mg/kg/d doses of BA in vehicle every second day for 14 days (7 doses). The mice were weighed, and tumor areas were measured throughout the study. After 22 days, the animals were sacrificed, final body and tumor weights were determined and selected tissues were further examined by routine hematoxylin and eosin staining and immunohistochemical analysis.

Tissue sections (4-5 μM thick) mounted on poly-Lysine-coated slides were deparaffinized by standard methods. Endogenous peroxidase was blocked by the use of 3% hydrogen peroxide in PBS for 10 min. Antigen retrieval for VEGF and CD31 staining was done for 10 min in 10 mmol/L sodium citrate buffer (pH 6) heated at 95° C. in a steamer followed by cooling for 15 min. The slides were washed with PBS and incubated for 30 min at room temperature with a protein blocking solution (VECTASTAIN Elite ABC kit, Vector Laboratories, Burlingame, Calif.). Excess blocking solution was drained and the samples were incubated overnight at 4° C. with one of the following: a 1:100 dilution of VEGF antibody or a 1:40 dilution of CD31 antibody. Sections then were incubated with biotinylated secondary antibody followed by streptavidin (VECTASTAIN Elite ABC kit). The color was developed by exposing the peroxidase to diaminobenzidine reagent (Vector Laboratories), which forms a brown reaction product. The sections were then counterstained with Gill's hematoxylin. VEGF and CD31 expression was identified by the brown cytoplasmic staining. Hematoxylin and eosin staining was determined as previously described [5].

### Antiproliferative, Proapoptotic and Antiangiogenic Effects

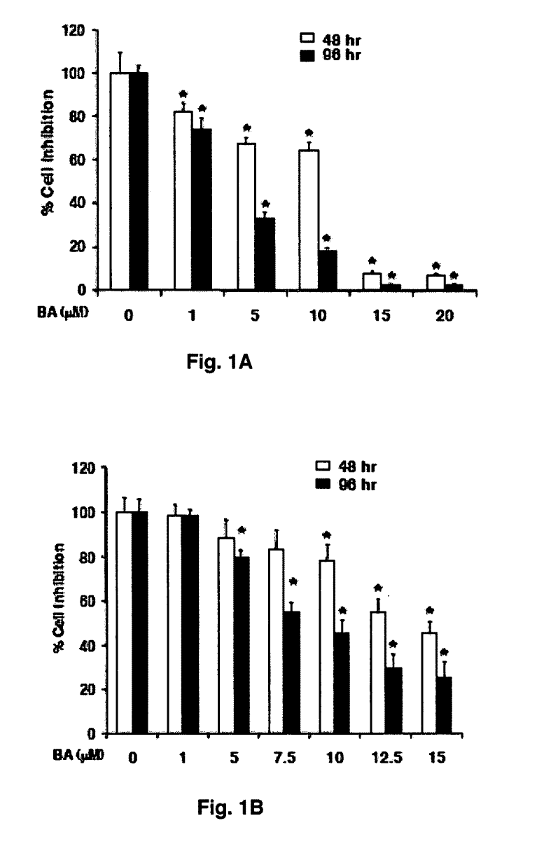

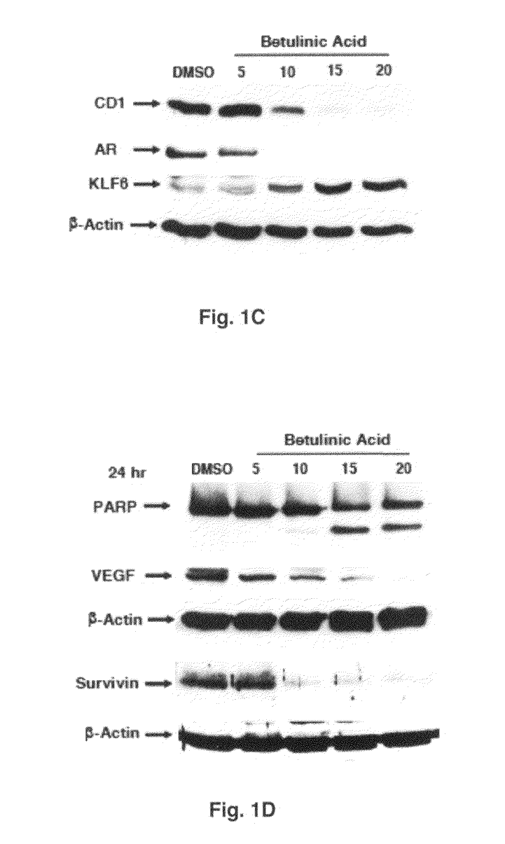

The antiproliferative effects of betulinic acid were initially investigated in SK-MEL2 melanoma and LNCaP prostate cancer cells. 6-day IC50 values for growth inhibition were 5-10 μM and 1-5 μM, respectively. Similar results were observed for pancreatic (Panc28, L3.6pl), bladder (KU7), and colon (SW480) cancer cells (data not shown). Cell survival curves for LNCaP and SK-MEL2 cells after treatment for 48 or 96 hr are illustrated in FIGS. 1A-1B. At higher concentrations of betulinic acid (≧10 μM), there was an overall decrease in the number of cells remaining compared to the number of initially seeded cells which was consistent with the cytotoxicity of betulinic acid.

The effects of betulinic acid on growth inhibitory and proapoptotic proteins were determined using LNCaP cells as a model. FIG. 1C shows that relatively short term exposure (24 hr) to BA (≧10 μM) induced downregulation of cyclin D1, whereas the cyclin-dependent kinase inhibitors p21 and p27 were expressed at low levels in these cells and were not affected by the treatment (data not shown). AR expression in LNCaP cells was decreased after treatment with 5 μM betulinic acid and this protein was almost completely absent in cells treated with 10 μM concentrations.

It has been demonstrated that AR knockdown by RNA interference in LNCaP cells resulted in induction of apoptosis [6], suggesting that the proapoptotic effects of betulinic acid in LNCaP cells may be due, in part, to the effects on AR expression. However, this mechanism would not be applicable to melanoma and other cancer cell lines which are AR-independent. The tumor suppressor gene KLF6 was also induced by betulinic acid at concentrations between 10-15 μM. BA also induced caspase-dependent PARP cleavage in LNCaP cells and this was accompanied by decreased expression of the antiapoptotic protein survivin (FIG. 1D) and induction of DNA laddering typically observed in cells undergoing apoptosis. In addition, betulinic acid also decreased expression of the angiogenic protein VEGF.

### BA Induces Proteosome-Dependent Degradation of Sp Proteins

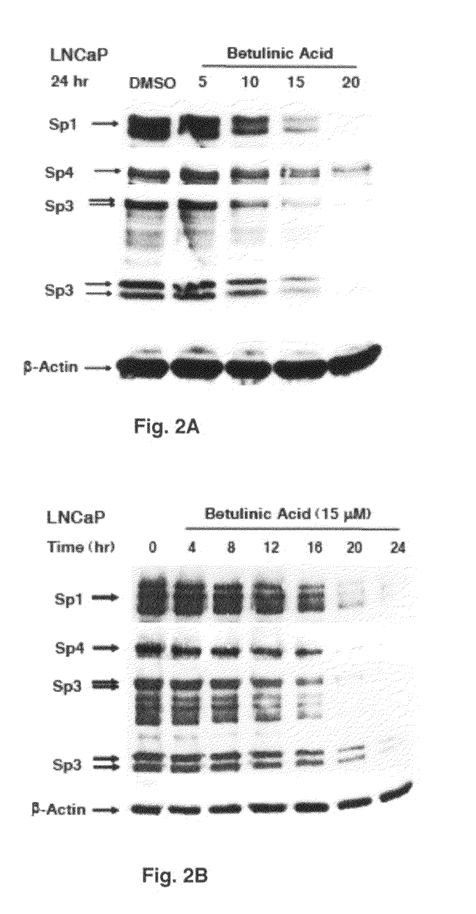

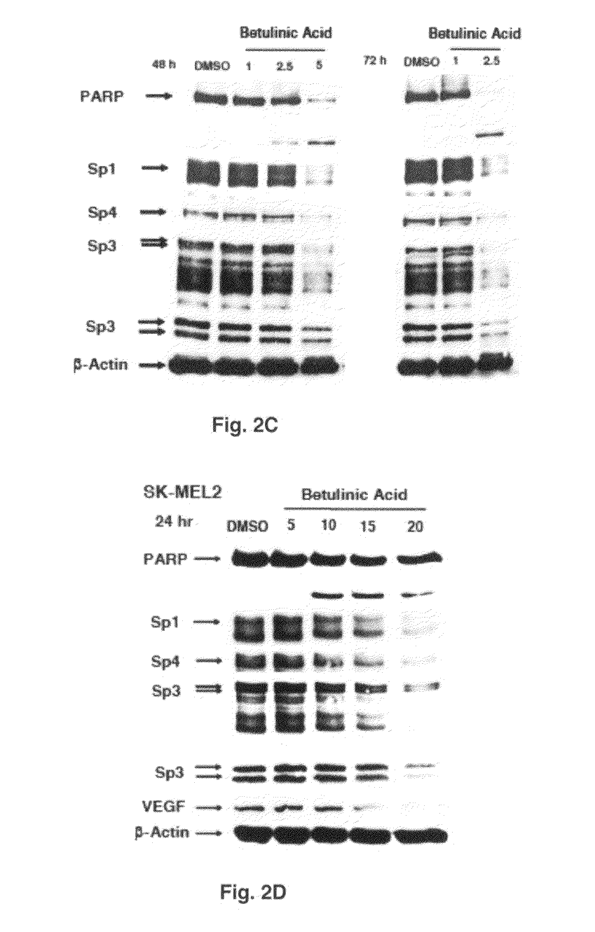

Results in FIG. 2A show that treatment of LNCaP cells with 5-10 μM betulinic acid for 24 hr induced downregulation of Sp1, Sp3 and Sp4 proteins. This also was accompanied by decreased expression of VEGF and survivin (FIG. 1D). The time-dependent decrease of Sp proteins in LNCaP cells treated with 15 μM betulinic acid for 4, 8, 12, 16, 20 and 24 hr showed that lower expression of these proteins is first observed after treatment for 12 hr (FIG. 2B). Prolonged treatment of LNCaP cells with betulinic acid for 48 or 72 hr showed that Sp protein degradation and PARP cleavage can be observed as doses as low as 1 to 2.5 μM (FIG. 2C). FIG. 2D illustrates that betulinic acid also decreased expression of Sp1, Sp3 and Sp4 proteins in SK-MEL2 melanoma cancer cells. Similar results were obtained in other cancer lines (data not shown).

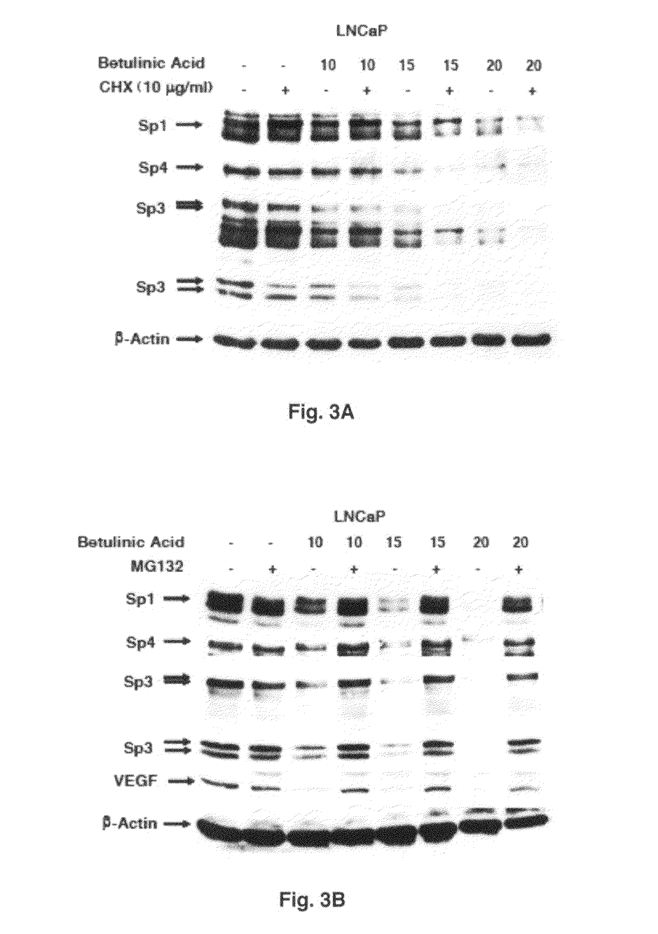

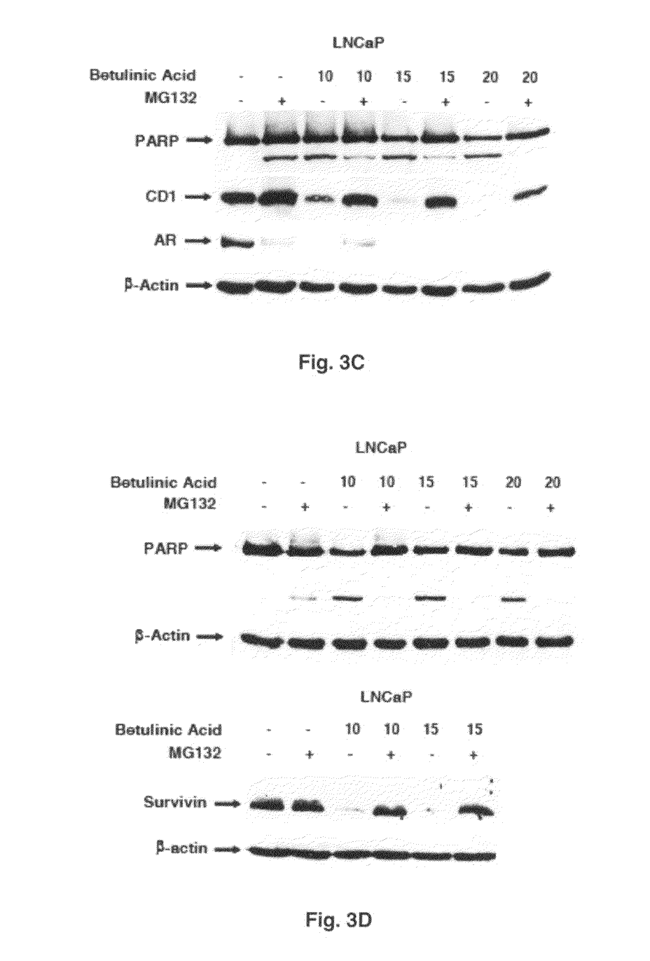

Since betulinic acid induces degradation of Sp proteins within 12 hr after treatment (FIG. 2B), the possible role of induced protein(s) in mediating this response was examined by treating LNCaP cells with betulinic acid alone (10-20 μM) or in the presence of 10 μg/ml cycloheximide (FIG. 3A). The protein synthesis inhibitor did not modulate the effects of betulinic acid on Sp protein levels. It has been demonstrated that COX-2 inhibitors, the NSAID tolfenamic acid and related compounds induced proteasome-dependent degradation of Sp proteins [5,7]. Thus, the effects of the proteasome inhibitor MG132 on betulinic acid-induced downregulation of Sp1, Sp3, Sp4 and VEGF in LNCaP cells was examined. MG132 reversed the effects of betulinic acid on expression of these transcription factors (FIG. 3B). BA selectively induced proteasome-dependent degradation of Sp proteins and cyclin D1 in LNCaP cells (FIG. 3C); however, MG132 did not modulate expression of β-actin or reverse betulinic acid-dependent downregulation of AR which is due to decreased AR RNA expression (data not shown). MG132 (10 μM) and other proteasome inhibitors induced caspase-dependent cleavage of PARP in LNCaP cells; however, MG132 also partially inhibited betulinic acid-induced apoptosis. Moreover, using a lower concentration of MG132 (5 μM), the effects of the compound alone on PARP cleavage were decreased but in combination with betulinic acid, there was inhibition of betulinic acid-induced PARP cleavage (FIG. 3D). MG132 also blocked betulinic acid-induced downregulation of survivin protein (FIG. 3D), suggesting that Sp protein degradation plays a role in the apoptosis-inducing effects of BA.

### BA Inhibits VEGF and Survivin Promoter Expression in LNCaP Cells

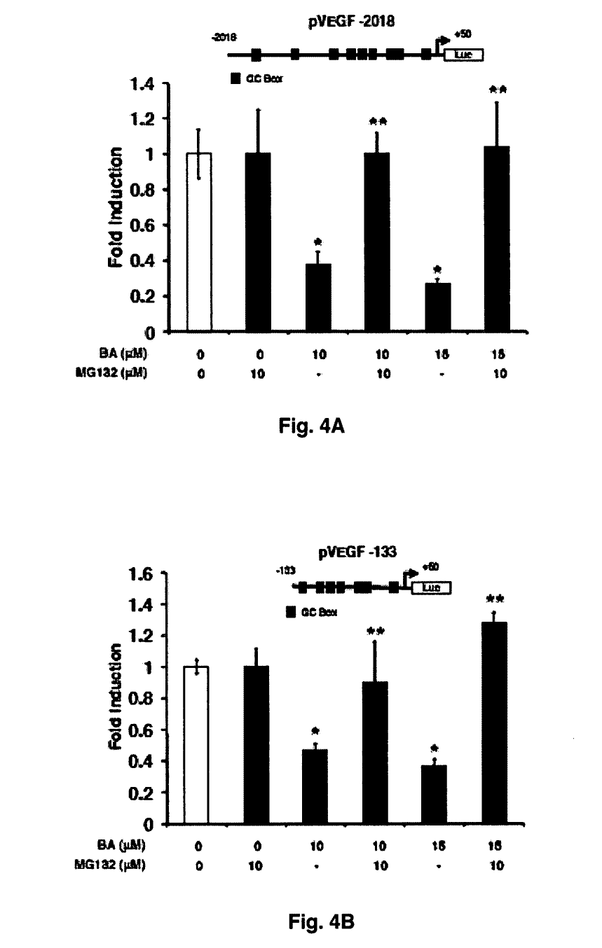

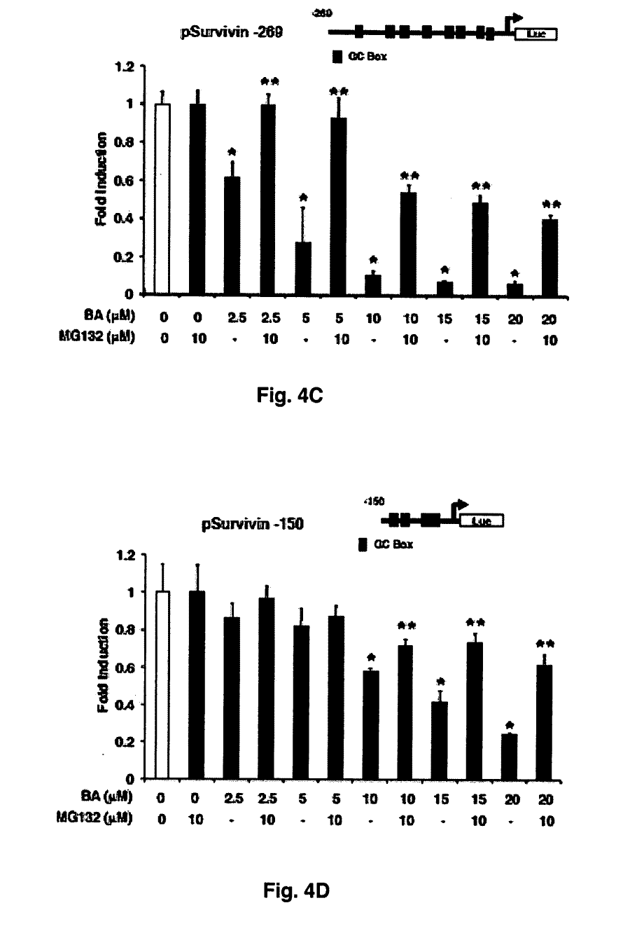

The effects of betulinic acid on decreased expression of VEGF and survivin through Sp protein degradation was examined further in transfection studies. The effects of betulinic acid on transactivation was investigated in LNCaP cells transfected with the pVEGF1 and pVEGF2 constructs containing the −2068 to +50 and −133 to +50 VEGF gene promoter inserts. FIGS. 4A-4B show that betulinic acid decreased luciferase activity in LNCaP cells transfected with pVEGF1 and pVEGF2 and that these effects were reversed by the proteasome inhibitor MG132. Similar results were observed using the proteasome inhibitor lactacystin (data not shown). This further confirms that betulinic acid induced degradation of Sp proteins results in decreased VEGF expression in LNCaP cells. This is consistent with previous RNA interference studies demonstrating that Sp1, Sp3 and Sp4 regulate VEGF expression in cancer cell lines (4 22).

Since the proteasome inhibitor MG132 partially blocks betulinic acid-induced PARP cleavage (FIGS. 3C-3D), the role of Sp protein degradation on induction of apoptosis was examined. The effects of betulinic acid on transactivation in LNCaP cells transfected with the GC-rich pSurvivin-269 and pSurvivin-150 constructs containing the −269 to +49 and −150 to +49 survivin promoter inserts were determined. Betulinic acid causes a concentrationdependent decrease in luciferase activity which was significantly reversed by 10 μM MG132 (FIGS. 4C-4D). Electrophoretic mobility shift assays also show that lysates from betulinic acid-treated LNCaP cells exhibited decreased binding to GC-rich survivin sequences.

### BA-Induced Sp Proteins Degradation in Pancreatic Cancer

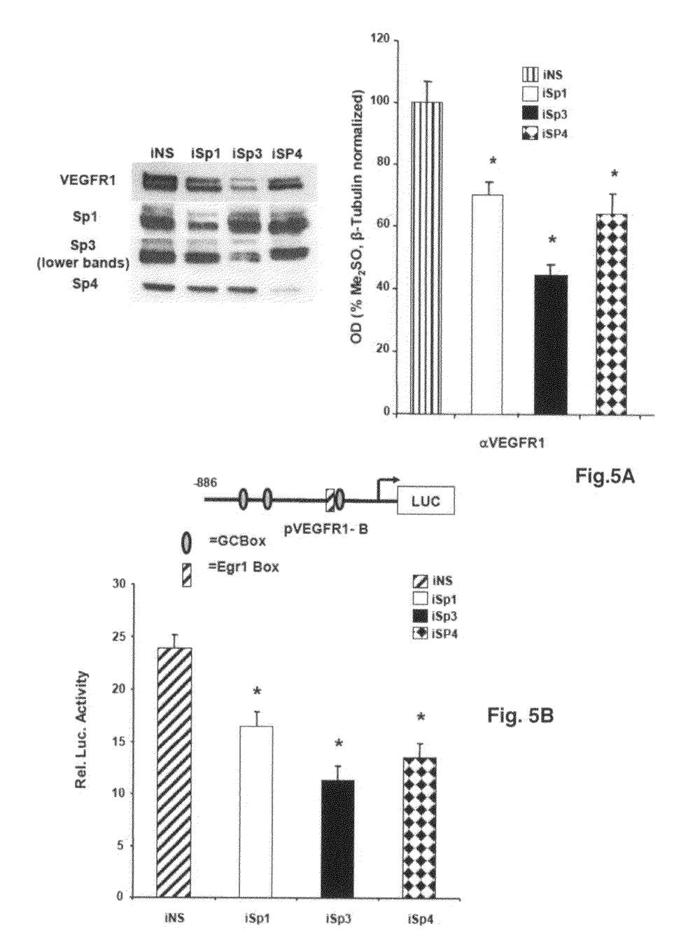

Pancreatic cancer cells (Panc-1) transfected with small inhibitory RNAs for Sp1 (iSp1), Sp3 (iSp3) and Sp4 (iSp4) exhibit a decrease in expression of Sp1 proteins accompanied by a decrease in expression of VEGFR1 protein (FIG. 5A). RNA interference also decreases transactivation in Panc-1 cells transfected with a series of VEGFR1 constructs (FIG. 5B).

### Example 3

### BA Inhibits Tumor Growth

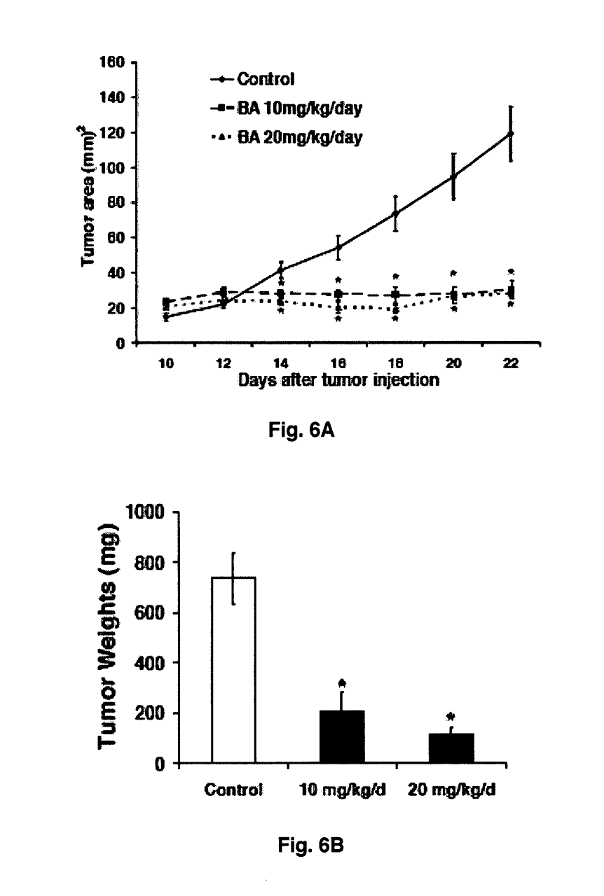

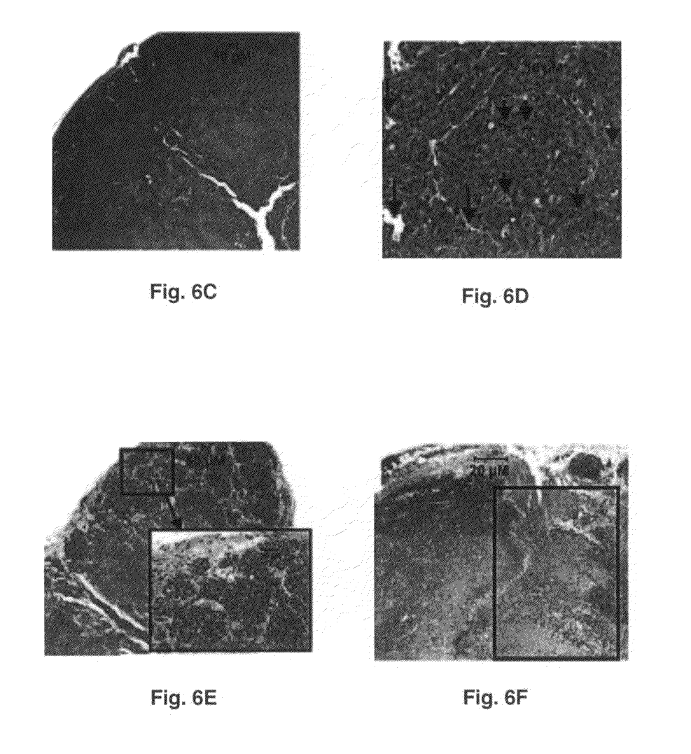

FIG. 6A shows that 10 and 20 mg/kg/d betulinic acid inhibited tumor growth in mice bearing LNCaP cell xenografts and that this was accompanied by significantly decreased tumor weights in both treatment groups (FIG. 6B). Examination of the mice showed that there were no treatment-related changes in organ or body weights or in the histopathology of liver and other tissues (data not shown). This was consistent with the reported low toxicity of this compound (1 20).

Representative hematoxylin- and eosin-stained histopathology sections of prostate tumors from the control and treated mice were examined. Tumors from untreated mice consisted of minimally encapsulated (FIG. 6C), dense expansile nests of epithelial cells with marked atypical features such as anisocytosis, anisokaryosis, multiple variably sized nucleoli, nuclear molding, bi- and multinucleation. Bizarre mitotic figures were frequently noted within the neoplastic cells (FIG. 6D, arrow heads). Abundant vascular channels were frequently present within neoplastic cells (FIG. 6D, arrows). Tumors from the treated mice consisted of neoplastic cells similar to that noted from the untreated mice. However, the mitotic activity (1-2 microfigures/hpf compared to 6-8 microfigures/hpf in the corn oil group) and the epithelial atypia appeared to be decreased (FIG. 6E). In addition, the tumor tissue was remarkably less vascular with evidence for necrosis (FIG. 6F, area within box), and this is consistent with the antiangiogenic effect of BA through decreased expression of Sp proteins and VEGF.

### BA Decreases Sp Protein and VEGF Expression in Tumors but not Liver

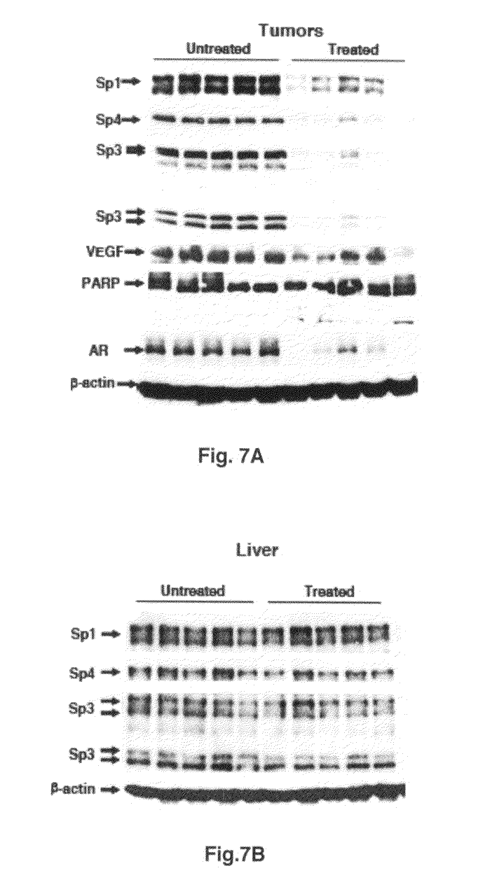

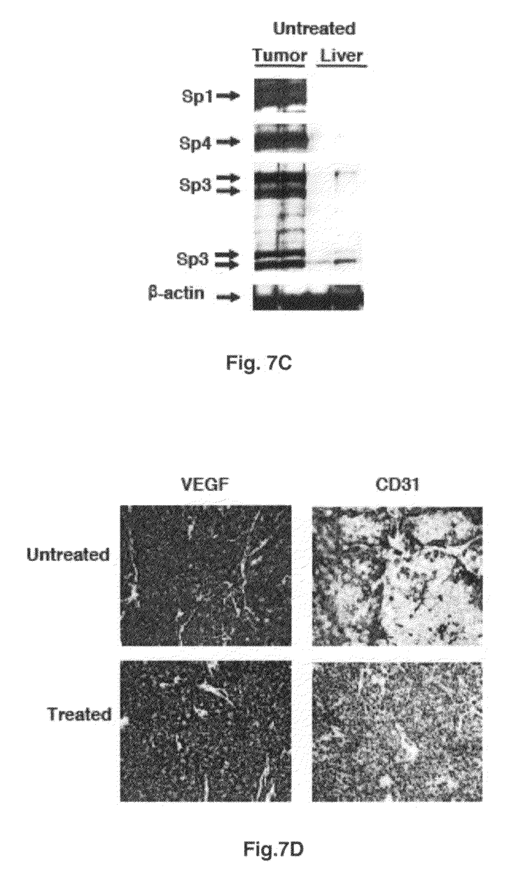

Expression of Sp proteins, AR and VEGF and PARP cleavage was compared in tumor lysates from control and BA-treated mice (5 animals per group) by Western blot analysis using β-actin as a loading control. Relatively high levels of Sp1, Sp3, Sp4 and VEGF proteins were observed in the control tumors while PARP cleavage in protein lysates from these tumors was not detected (FIG. 7A). In contrast, expression of Sp1, Sp3, Sp4, AR and VEGF proteins was decreased in tumors from BA-treated mice and PARP cleavage was observed. Sp protein levels in liver from untreated or BA-treated mice could be visualized only after prolonged exposures and the pattern of Sp1, Sp3 or Sp4 protein expression was similar in both groups (FIG. 7B). A direct comparison of Sp protein expression in tumors and liver from untreated animals (FIG. 7C) indicates high levels in tumors, whereas in liver only Sp3 could be detected and levels of Sp1 and Sp4 were very low.

Sp proteins in other tissues/organs from mice treated with corn oil or BA were examined and uniformly very low to non-detectable Sp1, Sp3 and Sp4 in both treatment groups was observed. Tumors from untreated and treated mice also were stained for VEGF and CD31 (microvessel density). The staining for both factors was decreased in tumors from BA-treated mice (FIG. 7D).

Therefore betulinic acid is a potent inhibitor of prostate cancer cell and tumor growth. The underlying mechanisms of proapoptotic, antiproliferative and antiangiogenic activities of betulinic acid is associated with induction of proteasome-dependent degradation of Sp proteins in prostate tumor cells. Moreover, Sp protein expression was decreased in SK-MEL2 (FIG. 2D) and other cancer cell lines. Thus it is contemplated that BA-dependent downregulation of Sp proteins which is accompanied by both proapoptotic and antiangiogenic responses is an integral part of the anticarcinogenic activity of this compound.

### Example 4

### CN-BA and CN-BA-Me Modulate PPARγ in Colon and Pancreatic Cancers

SW480, HT-29 and HCT-15 human colon cancer cells were provided by Dr. Stan Hamilton (M. D. Anderson Cancer Center (Houston, Tex.). Panc-28 human pancreatic cancer cells and 3T3-L1 pre-adipocytes were obtained from American Type Culture Collection (Manassas, Va.). SW480, HT-29 and Panc-28 cells were maintained in Dulbecco's modified/Ham's F-12 (Sigma-Aldrich, St Louis, Mo.) with phenol red supplemented with 0.22% sodium bicarbonate, 0.011% sodium pyruvate, 5% fetal bovine serum, and 10 mL/L 100× antibiotic antimycotic solution (Sigma). HCT-15 cells were maintained in RPMI 1640 (Sigma) supplemented with 0.22% sodium bicarbonate, 0.011% sodium pyruvate, 0.45% glucose, 0.24% HEPES, 10% fetal bovine serum, and 10 mL/L of 100× antibiotic antimycotic solution (Sigma). Cells were maintained at 37° C. in the presence of 5% CO₂.

Reporter lysis buffer and luciferase reagent for luciferase studies were supplied by Promega (Madison, Wis.). β-Galactosidase (β-Gal) reagent was obtained from Tropix (Bedford, Mass.), and LipofectAMINE reagent was purchased from Invitrogen (Carlsbad, Calif.). The PPARγ antagonist N-(4′-aminopyridyl)-2-chloro-5-nitrobenzamide (T007) [8] was synthesized in this laboratory, and its identity and purity (>98%) was confirmed by gas chromatography-mass spectrometry.

### Cell Proliferation Assay

This assay is performed in 12-well tissue culture plates at the concentration of 2×10⁴ cells per well, using DMEM/Ham's F-12 media containing 2.5% charcoal stripped FBS. The cells were counted on the initial day using a Z1 cell counter (Beckman Coulter, Fullerton, Calif.) and then were treated either with vehicle (DMSO) or the indicated triterpenoid compounds, each sample in triplicate. Every 48 hr, fresh medium was added along with the indicated compounds. The count of the cells was taken after 2, 4 and 6 days. The results are expressed as means±S.E for each set of triplicate.

### Mammalian Two Hybrid Assay

The GAL4 reporter construct containing 5×GAL4 response elements (p GAL4) was provided by Dr. Marty Mayo (University of North California, Chapel Hill, N.C.). The GAL4-coactivator fusion plasmids pM-SRC1, pMSRC2, pMSRC3, pM-DRIP205, pM-CARM-1 and PPARγ-VP16 fusion plasmid (Vp-PPARγ) containing the DEF region of the PPARγ (amino acids 183-505) fused to the pVP16 expression vector were provided by Dr. Shigeaki Kato (University of Tokyo, Tokyo, Japan). SW480 colon cancer cells were plated in 12-well tissue culture plates at 1×10⁵ cells per well in DMEM/Ham's F-12 medium supplemented with 2.5% charcoal stripped FBS.

After allowing them to adhere overnight, transient transfections were carried out with GAL4-Luc (0.4 μg), β-GAL (0.04 μg), VP-PPARγ (0.04 μg), pM-SRC1 (0.04 μg), pM-PGC-1 (0.04 μg), pM-SMRT (0.04 μg), pM-TRAP220 (0.04 μg), pM-DRIP205 (0.04 μg), and pMCARM1 (0.04 μg) using LipofectAMINE2000 (Invitrogen) following the manufacturer's guidelines. After 6 hr of transfection, cells were treated in triplicate either with vehicle (DMSO) or the indicated compound suspended in complete medium for 20-24 hr. One hundred μL per well of 1× Reporter Lysis Buffer (Promega) was used to lyse the cells and 30 μl of this lysate was used to perform the luciferase and β-GAL assays using Lumicount (Perkin-Elmer Life and Analytical Sciences, Boston, Mass.). The luciferase activities obtained were normalized to the β-gal activity.

### Transfections

Cells were seeded onto the 12-well plates and 0.4 μg of GAL4-Luc, 0.04 μg of β-GAL, 0.04 μg of GAL4DBD-PPARγ, and 0.4 μg of p21-luc(FL) containing −2325 to +8 insert, 0.4 μg of p21-luc (−124) containing −124 to +8 insert and 0.4 μg of p21-LUC (−60) containing −60 to +8 insert were transfected using LipofectAMINE reagent (Invitrogen) following the manufacturer's protocol. Cells were treated either with vehicle or respective compounds suspended in complete medium after 6 hr of transfection. Cell lysate is extracted after 20-22 hr by adding 100 μl of 1× reporter lysis buffer per well and 30 μl of this extract is used to quantitate the luciferase activity using Lumicount (Perkin-Elmer Life and Analytical Sciences). Each experiment is done in triplicate and the results are normalized to the β-GAL activity.

### Western Blot Analysis

SW480, HT-29, HCT-15 and Panc-28 (3×10⁵) colon cancer cells were seeded in 6-well tissue culture plates in DMEM/Ham's F-12 medium containing 2.5% charcoal-stripped FBS. Protein is extracted from the cells treated either with vehicle or indicated compounds suspended for 24 hr except for caveolin-1 protein which was done for 72 hr. Samples were extracted in high salt buffer [50 mmol/L HEPES, 500 mmol/L NaCl, 1.5 mmol/L MgCl₂, 1 mmol/L EGTA, 10% glycerol, and 1% Triton X-100 (pH 7.5), and 5 μL/mL protease inhibitor cocktail (Sigma-Aldrich)]. Samples were incubated at 100° C. for 2 min, separated on either 10% or 12% SDS-PAGE gels and then transferred to polyvinylidene difluoride membrane (PVDF; Bio-Rad, Hercules, Calif.). The PVDF membrane was blocked in 5% TBST-Blotto (10 mM Tris HCl, 150 mM NaCl, pH 8.0, 0.05% Triton X-100, and 5% nonfat dry milk) for about 30 min and was then incubated in fresh 5% TBST-Blotto with 1:1000 for caveolin-1 (Santa Cruz Biotechnology, Santa Cruz, Calif.), 1:1000 for p21 (BD Pharmingen, Frank lakes, NJ) and 1:10000 for β-actin (Sigma) primary antibody overnight with gentle shaking at 4° C. After washing with TBST for 10 min, the membrane was incubated with respective secondary antibody (1:5000) (Santa cruz, Calif.) in 5% TBST-Blotto for 3 hr. The membrane is then washed with TBST for 10 min, incubated with chemiluminiscence reagent from Perkin Elmer for one min and then exposed to Kodak X-OMAT AR autoradiography film (Eastman Kodak, Rochester, N.Y.).

### Differentiation and Oil Red O Staining

3T3-L1 preadipocytes were cultured on poly-lysine-coated coverslips with DMEM and 10% FBS at 5% CO₂ in 24-well plates. At 2 days post-confluence, cells were incubated with fresh media supplemented with 3-isobutyl-1-methylxanthine (0.5 mM), dexamethasone (1 μM), insulin (1.7 μM), and indicated compounds (0.25 μM). After 48 hr, cells were changed to fresh media and treated with DMSO or indicated compounds for 5 days. Cells without any treatment for the entire 7 days were used as control. The cells were then fixed with 10% formalin. Fixed cells were washed with 60% isopropanol and stained with filtered 60% Oil Red O in deionized water. After staining, cells were washed with water and photographed.

### Semi Quantitative RT-PCR

SW480 and HT29 colon cancer cells were treated either with vehicle (DMSO) or indicated compounds and after 24 hr total RNA was extracted using RNeasy Mini kit (Qiagen, Inc., Valencia, Calif.). RNA concentration was measured by UV 260:280 nm absorption ratio and 2 μg RNA was used to synthesize cDNA using Reverse Transcription System (Promega). PCR conditions were as follows: initial denaturation at 94° C. (1 min) followed by 28 cycles of denaturation for 30 s at 94° C., annealing for 60 s at 55° C. and extension at 72° C. for 60 s and a final extension step at 72° C. for 5 min. The mRNA levels were normalized using GAPDH as an internal housekeeping gene. Primers obtained from IDT and used for amplification are as follows: KLF4 (sense 5′-CTA TGG CAG GGA GTC CGC TCC-3′ (SEQ ID NO: 1); antisense 5′-ATG ACC GAC GGG CTG CCG TAC-3′ (SEQ ID NO: 2)) and GAPDH (sense 5′-ACG GAT TTG GTC GTA TTG GGC G-3′ (SEQ ID NO: 3); antisense 5′-CTC CTG GAA GAT GGT GAT GG-3′ (SEQ ID NO: 4). PCR products were electrophoresed on 1% agarose gels containing ethidium bromide and visualized under UV transillumination.

### BA, CN-BA & CN-BA-Me Inhibit Cell Proliferation of SW480 and Panc-28 Cells

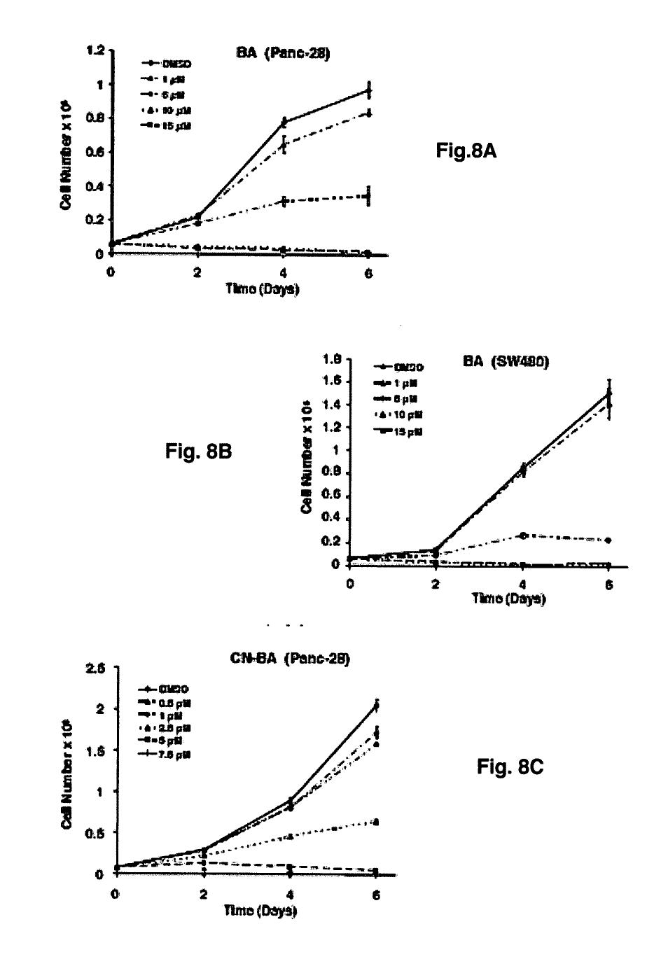

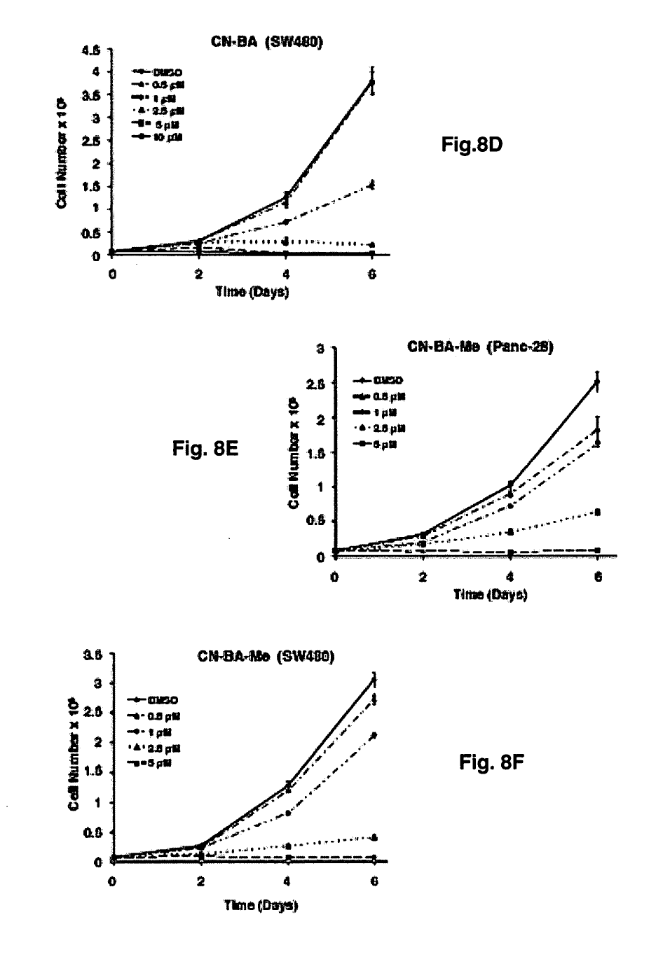

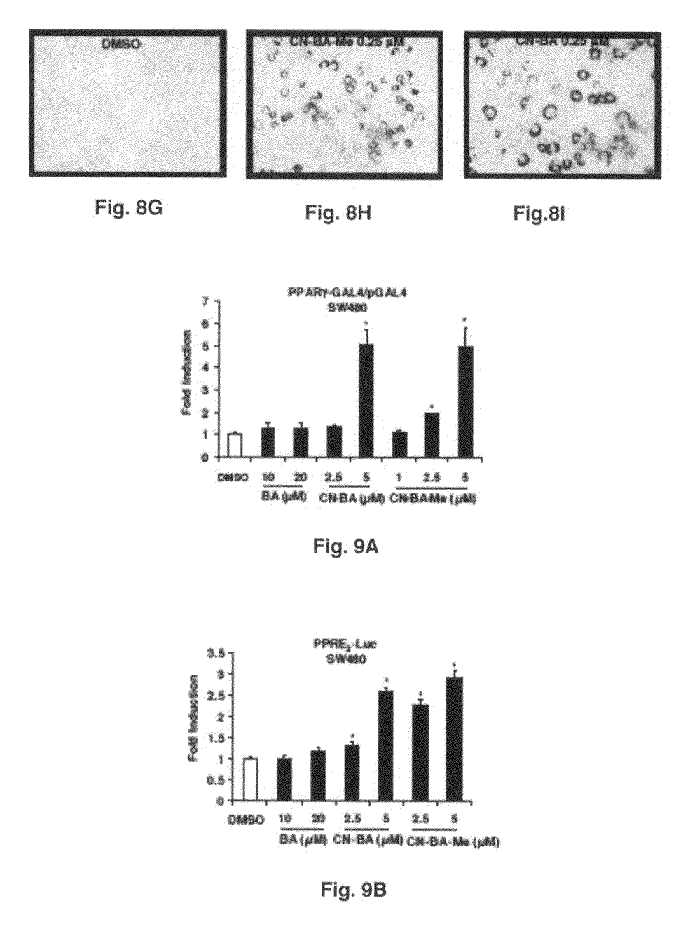

FIGS. 8A-8F illustrates the effects of betulinic acid, CN-BA and the corresponding methyl ester (CN-BA-Me) on growth of SW480 and Panc-28 cells. All three compounds inhibit growth of both cell lines and IC₅₀ values ranging from 1-5, 1-2.5 and 1-2.5 μM (Panc-28) and 1-5, 1.0 and 1-2.5 μM (SW480) were observed for betulinic acid, CN-BA and CN-BA-Me, respectively. CN-BA was the most cytotoxic compound in both cell lines and this confirms results of a previous report showing that 2-cyano derivatives of betulinic acid enhanced cytotoxicity [2]. One of the hallmarks of PPARγ agonists is their induction of differentiation in 3T3-L1 adipocytes which is characterized by accumulation of fat droplets which can be detected by Oil Red O staining. FIGS. 8G-8I show that both CN-BA and CN-BA-Me induce Oil Red O staining in this assay, whereas betulinic acid does not induce this response. These results suggest that these 2-cyano derivatives of betulinic acid exhibit activity associated with PPARγ agonists.

### CN-BA and CN-BA-Me Act as PPARγ Agonists

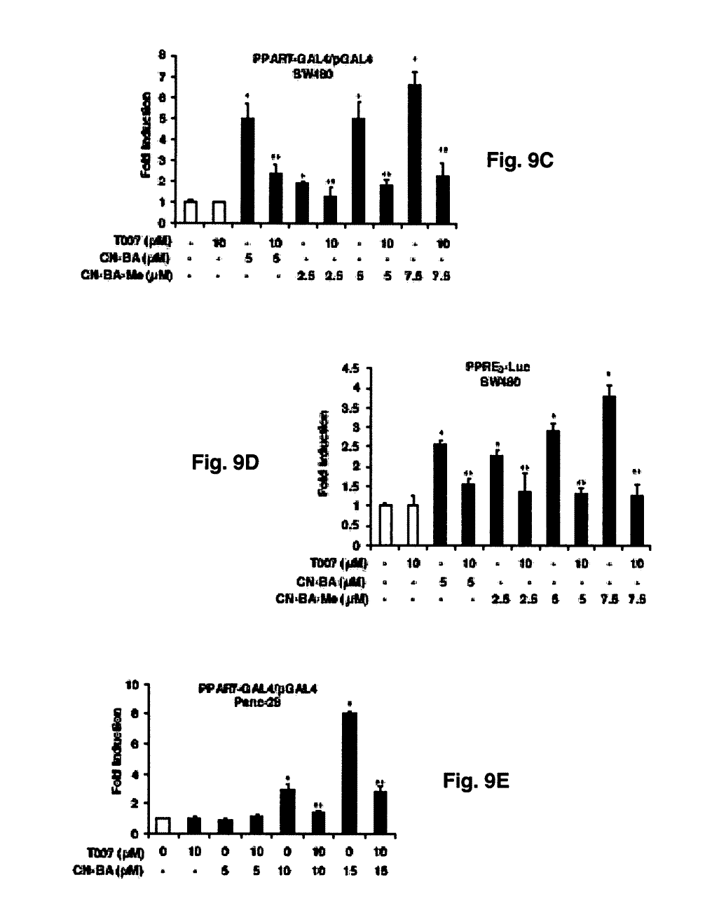

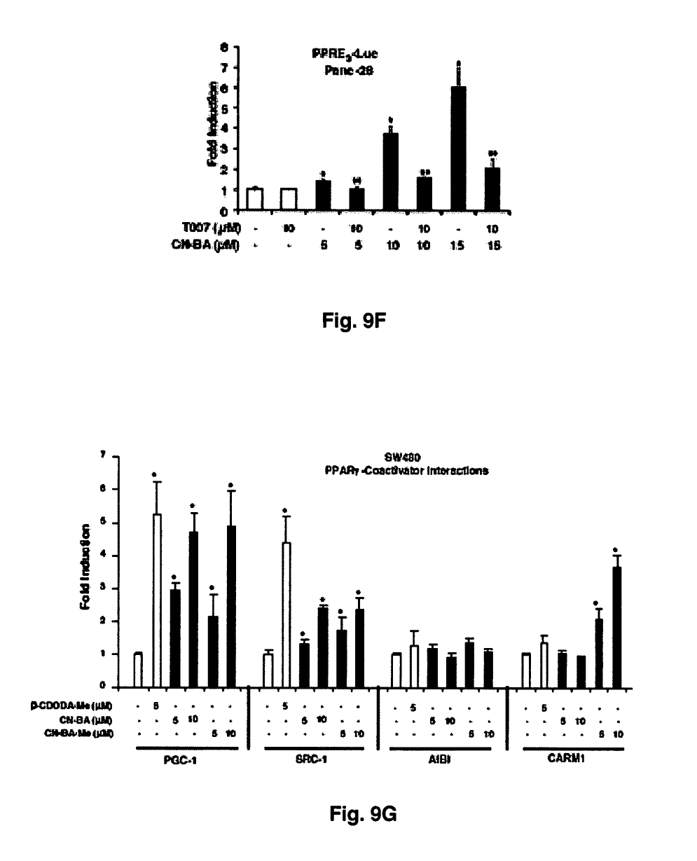

The PPARγ agonist activity of betulinic acid and related compounds was determined in SW480 cells transfected with PPARγ-GAL4/pGAL4 and a PPRE₃-luc construct (FIGS. 9A-9B). The results show that 2.5-10 μM CN-BA and CN-BA-Me induced transactivation, whereas betulinic acid was inactive in this assay. The PPARγ agonist activities also were determined in SW480 cells using the same constructs, but treated with CN-BA, CN-BA-Me alone or in combination with the PPARγ antagonist T007, and in all cases, the induced activities were inhibited by T007 (FIGS. 9C-9D). A similar approach was used in Panc-28 cells transfected with PPARγ-GAL4/pGAL4 and PPRE₃-luc (FIGS. 9E-9F) and CN-BA induced luciferase activity that was inhibited in cells cotreated with CN-BA plus T007.

Not surprisingly, betulinic acid was inactive in these assays; however, results obtained for CN-BA-Me were highly inconsistent in Panc-28 cells compared to the colon cancer cell line (FIGS. 9A-9D). CN-BA-Me exhibited minimal induction in cells transfected with PPARγ-GAL4/pGAL4 and no induction was observed in Panc-28 cells transfected with PPRE₃-luc (data not shown). These results were observed in replicate experiments suggesting that there were structure-dependent differences (CN-BA vs. CN-BA-Me) for activation of the PPARγ-GAL4/pGAL4 or a PPRE₃-luc constructs in Panc-28, but not SW480, cells.

FIG. 9G summarizes the effects of CN-BA and CN-BA-Me on induction of luciferase activity in SW480 cells transfected VP-PPARγ and GAL4-co-activator and GAL4 SMRT (a co-repressor) expression plasmids. β-CDODA-Me, a triterpenoid methyl ester derivative which also contains a 2-cyano-1-en-3-one function and activates PPARγ in colon cancer cells [9], as a comparative reference compound for the mammalian two-hybrid assay. The results show that CN-BA, CN-BA-Me and β-CDODA-Me significantly induced luciferase activity in SW480 cells transfected with VP-PPARγ and GAL4 PGC-1 and GAL4 SRC-1, but not GAL4-AIB1, GAL4-TIFII, GAL4-TRAP220 and GAL4-SMRT. In contrast, only CN-BA-Me also activated GAL4-CARM1 indicating differences between CN-BA and CN-BA-Me in the mammalian two-hybrid assay, suggesting that even among these two acid-ester analogs, some tissue-specific selective PPARγ modulator activity might be expected. The data are consistent with the differences observed for CN-BA and CN-BA-Me in activation of transfected constructs in Panc-28 cells (FIGS. 9E-9F).

### CN-BA and CN-BA-Me Induce p21 Protein Expression in Panc-28 Cells

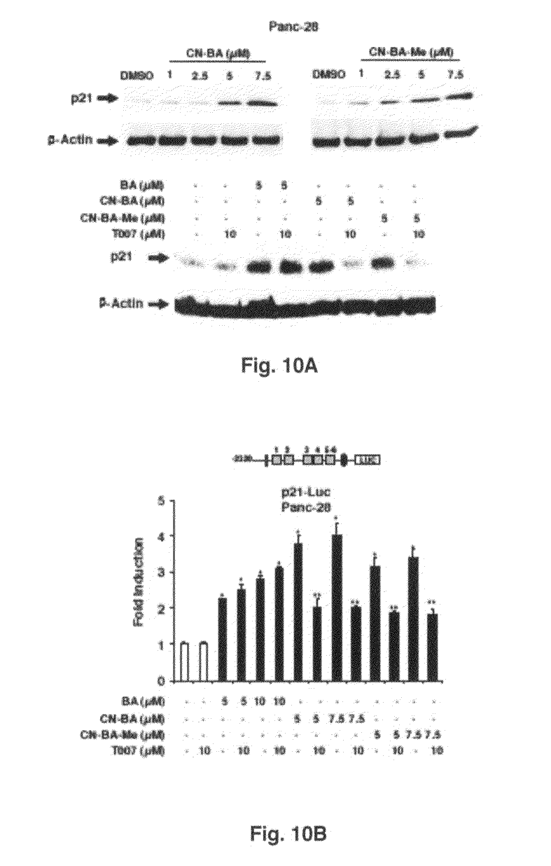

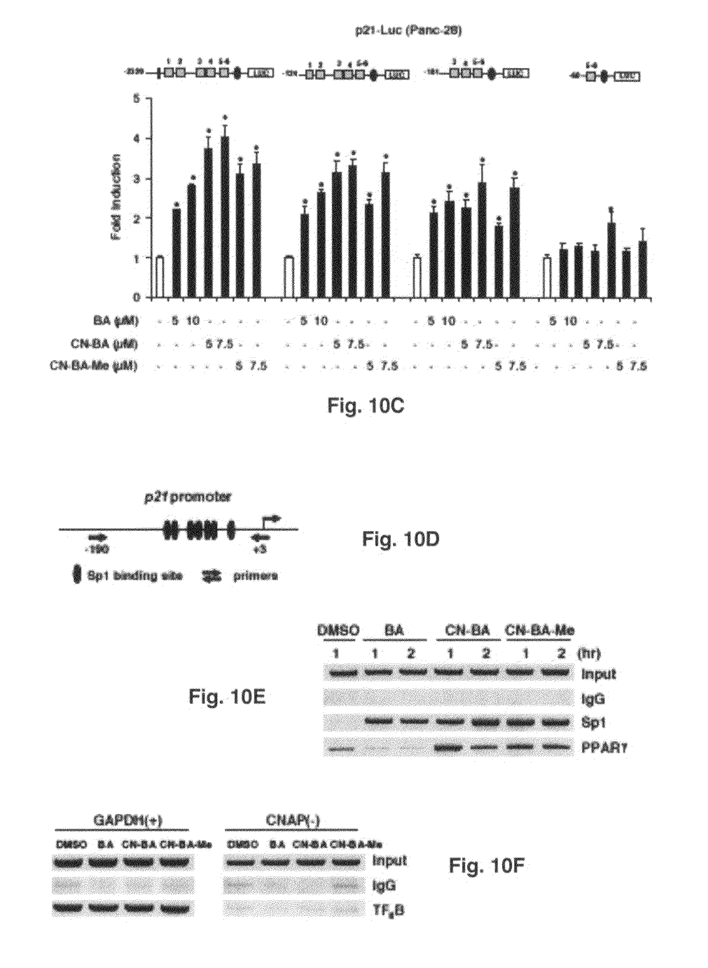

It has been demonstrated that PPARγ agonists induce p21 and p27 and decrease cyclin D1 expression in Panc-28 cells and only the former response is receptor dependent [10]. FIG. 10C shows that both CN-BA and CN-BA-Me induce p21 protein expression in Panc-28 cells and that this also is accompanied by induction of p27 and downregulation of cyclin D1, as previously reported for a series of PPARγ-active methylene-substituted diindolylmethanes (C-DIM) analogs in this cell line [10]. Cotreatment of Panc-28 cells with 5 μM CN-BA and CN-BA-Me plus the 10 μM T007 significantly inhibited induction of p21, confirming that induction of p21 was PPARγ-dependent (FIG. 10A). In contrast, induction of p21 by BA was not inhibited after cotreatment with T007 and this was consistent with results of transactivation studies showing that betulinic acid does not activate PPARγ in Panc-28 or SW480 (FIGS. 9A-9G).

FIG. 10B shows that betulinic acid, CN-BA and CN-BA-Me induce transactivation in Panc-28 cells transfected with p21-luc(Fl) which contains the −2325 to +8 region of the p21 promoter. In cells cotreated with betulinic acid and related compounds plus the PPARγ antagonist T007, the induction of luciferase activity by CN-BA and CN-BA-Me was inhibited, whereas BA-induced activity was unaffected by T007. The results complement the immunoblot data confirming that induction of p21 by CN-BA/CN-BA-Me was PPARγ-dependent, whereas induction of p21 by betulinic acid was PPARγ-independent.

Induction of luciferase activity in Panc-28 cells transfected with constructs containing −2325 to +8 [p21-Luc (Fl)], −124 to +8 [p21-Luc (−124)], −101 to +8 [p21-Luc (−101)], and −60 to +8 [p21-Luc (−60)] p21 promoter inserts was examined. The latter three constructs contain the 6 proximal GC rich sites (1-6) and the results of the transfection studies suggest that these GC-rich sites are necessary for CN-BA- and CN-BA-Me-induced transactivation. Deletion analysis of the p21 promoter indicated that loss of inducibility, i.e. p21-luc(60), was associated with loss of GC-rich sites 3 and 4, whereas CN-BA significantly induced activity but only at the 7.5 μM concentration. This suggests sites 3 and 4 were also important for this compound but induction could also be observed using constructs containing only GC-rich sites 1 and 2.

It has been demonstrated that PPARγ-dependent activation of p21 by other PPARγ agonists also is dependent on GC-rich sites 3 and 4 and involves PPARγ/Sp-dependent activation of p21. The ligand-dependent recruitment of PPARγ to the p21 promoter by CN-BA and CN-BA-Me was examined using a ChIP assay of Panc-28 cells treated with the betulinic acid, CN-BA and CN-BA-Me for 1 or 2 hr. FIGS. 10D-10F show that both CN-BA and CN-BA-Me recruited PPARγ to the proximal GC-rich region of the p21 promoter. This also was accompanied by enhanced binding of Sp1. In contrast, betulinic acid did not induce PPARγ interactions with the p21 promoter in the ChIP assay. This is consistent with receptor-independent activation of p21 by betulinic acid and the mechanism of this response is currently being investigated. As a control for this experiment, transcription factor TFIIb bound to the proximal region of the GAPDH gene promoter but not to exon-1 of the CNAP1 gene.

### CN-BA and CN-BA-Me Induce Caveolin-1 Expression in HT-29 Cells

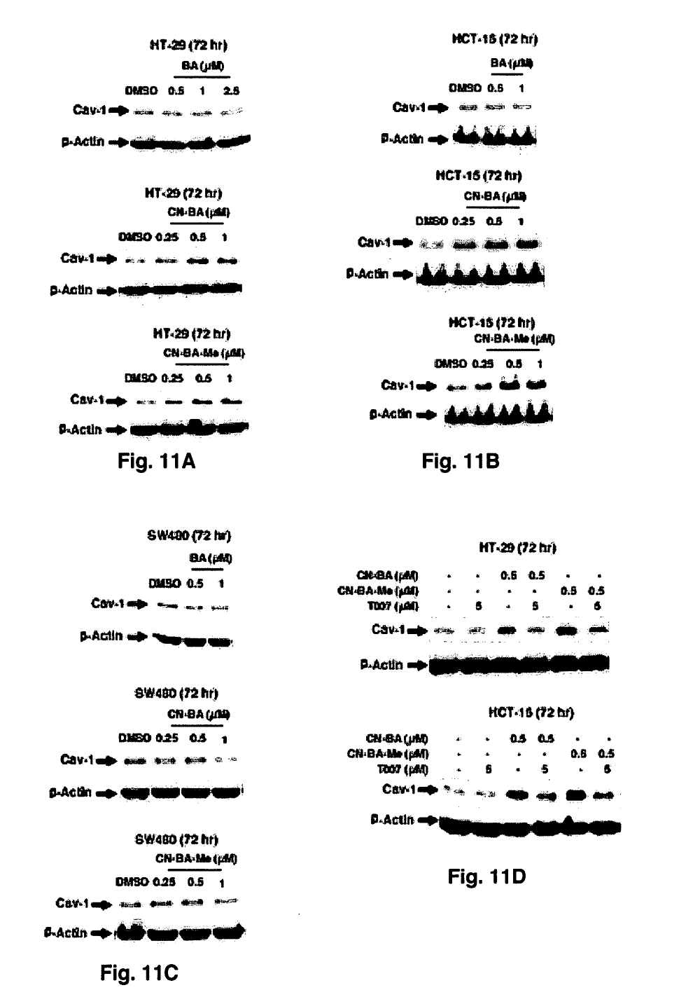

PPARγ agonists such as CDDO, β-CDODA and related esters and PPARγ-active C-DIMs also induce receptor-dependent expression of caveolin-1 in colon cancer cells [10-13]. FIG. 11A shows that CN-BA, CN-BA-Me, but not BA induce caveolin-1 in HT-29 cells. Similar results were observed in HCT-15 cells (FIG. 11B). In contrast, betulinic acid, CN-BA and CN-BA-Me did not induce caveolin-1 expression in SW480 cells and the latter two compounds decreased expression of this protein (FIG. 11C). Cotreatment of HT-29 and HCT-15 cells with CN-BA/CN-BA-Me plus the PPARγ antagonist T007 resulted in inhibition of the induced caveolin-1 response, confirming that induction was PPARγ-dependent (FIG. 11D-11E). Thus, receptor-dependent activation of caveolin-1 by CN-BA and CN-BA-Me was dependent on cell context. This correlates with CDDO and α-CDODA-Me, but not β-CDODA-Me, inducing caveolin-1 in HT-29 and SW480 cells, whereas, like CN-BA/CN-BA-Me, β-CDODA induced caveolin-1 in HT-29, but not in SW480 cells.

### CN-BA and CN-BA-Me Induce KLF4 Expression in HT-29, but not SW480, Cells

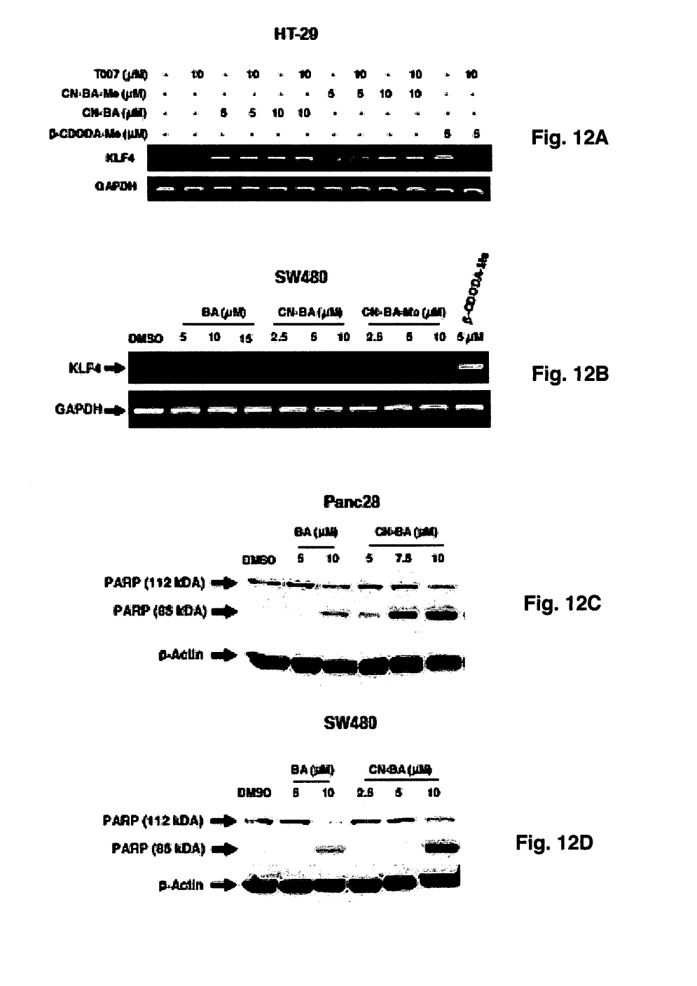

FIGS. 12A-12D summarize the effects of CN-BA and CN-BA-Me on KLF4 expression in HT-29 and SW480 cells. In KLF4 cells, CN-BA and CN-BA-Me induced KLF4 mRNA levels. Similar results were observed for β-CDODA-Me which was used as a positive control for this cell line. However, in HT-29 cells cotreated with CN-BA, CN-BA-Me and β-CDODA-Me plus the PPARγ antagonist T007, induction of KLF4 was decreased significantly only for β-CDODA-Me. In contrast, CN-BA and CN-BA-Me did not induce KLF4 expression in SW480 cells, whereas α-CDODA-Me treatment enhanced KLF4 mRNA [9].

The differences between CN-BA/CN-BA-Me and β-CDODA-Me as inducers of KLF4 mRNA levels in colon cancer cells clearly distinguished between two classes of structurally related PPARγ agonists derived from triterpenoid acids confirming that CN-BA/CN-BA-Me are a novel class of PPARγ antagonists. In addition, it is confirmed that BA/CN-BA induced apoptosis in SW480 and Panc28 cells and FIGS. 12C-12D show that both compounds induced caspase-dependent PARP cleavage in these cell lines.

### CN-BA Inhibition of Cell Proliferation In Vitro in LNCaP and Ku7 Cells

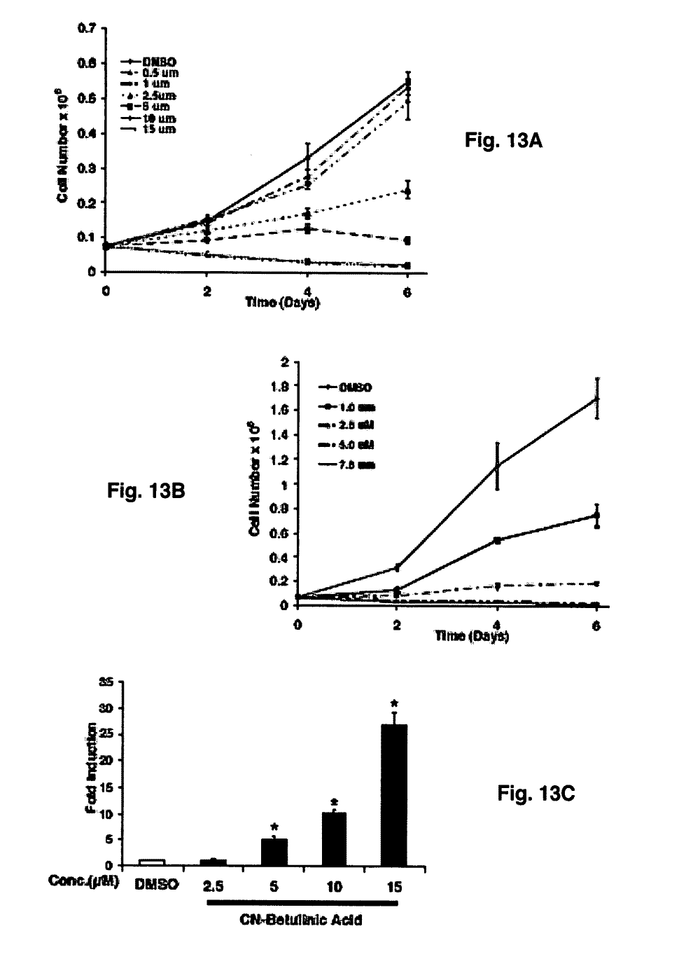

2-cyanobetulinic acid (CN-BA) inhibits growth of LNCaP prostate and Ku7 bladder cancer cells with IC50 values of approximately 2.5 & 1.0 M, (FIGS. 13A-13C). These derivatives are more cytotoxic than BA alone and which may be due to the PPARγ agonist activity of CN-BA which activates a PPARγ-GAL4 chimera whereas BA is inactive in this assay.

### Example 5

### BA, CN-BA and CN-BA-Me Effect Sp Protein Expression in Colon and Pancreatic Cancer Cells BA and CN-BA Induce Apoptosis in RKO Cells

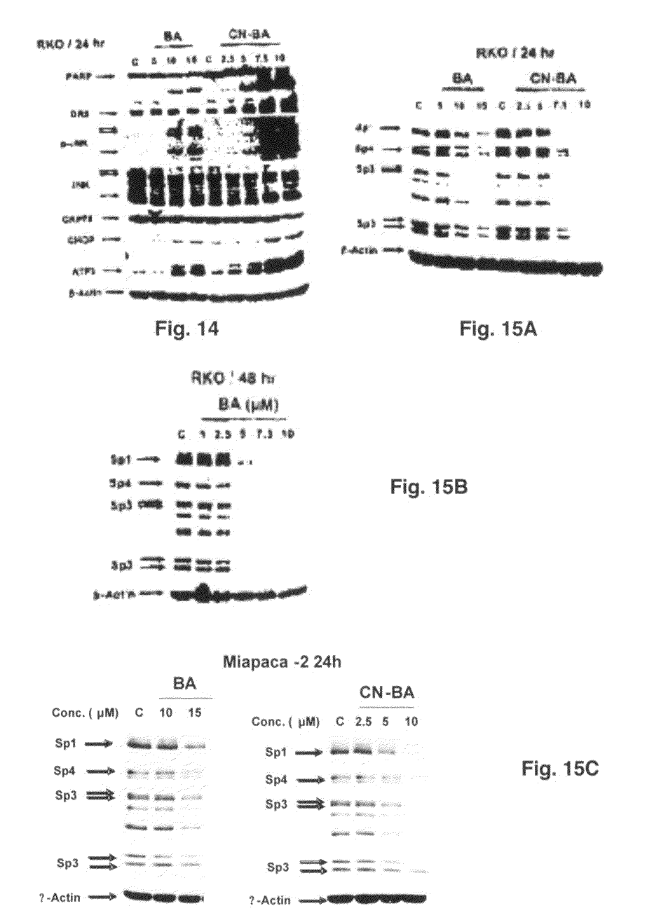

RKO cells were transfected with BA or CN-BA for 24 hours and analyzed by Western blot. FIG. 14 shows that both BA and CN-BA activate proapoptotic responses in RKO cells. In addition BA and CN-BA activate ATF3, JNK phosphorylation, CHOP, and DR5, but not CRP78.

### BA and CN-BA Decrease Sp Protein Expression in RKO Cells

The effects of BA and CN-BA on Sp protein expression in RKO colon cancer cells were examined. FIGS. 15A-15C shows that BA and/or CN-BA decrease Sp protein expression in RKO cells over 24 and 48 hr and in Miapaca-2 pancreatic cancer cells over 24 hr. Similar results are observed in SW480 and HT-29 colon cancer cells and in L3.6pl and Panc-28 pancreatic cancer cells (data not shown). In RKO cells treated with BA for 48 hr (FIG. 15B), Sp proteins are decreased by 1-5 μM concentrations of BA. This is in the same range of concentrations required for inhibition of cell growth and induction of apoptosis. CN-BA shows similar results. It is contemplated that decreased expression of Sp proteins by betulinic acid and/or its analogs and derivatives significantly contributes to their anticarcinogenic activities.

BA and CN-BA Modulates microRNA Transcription in Colon and Pancreatic Cancer Cells

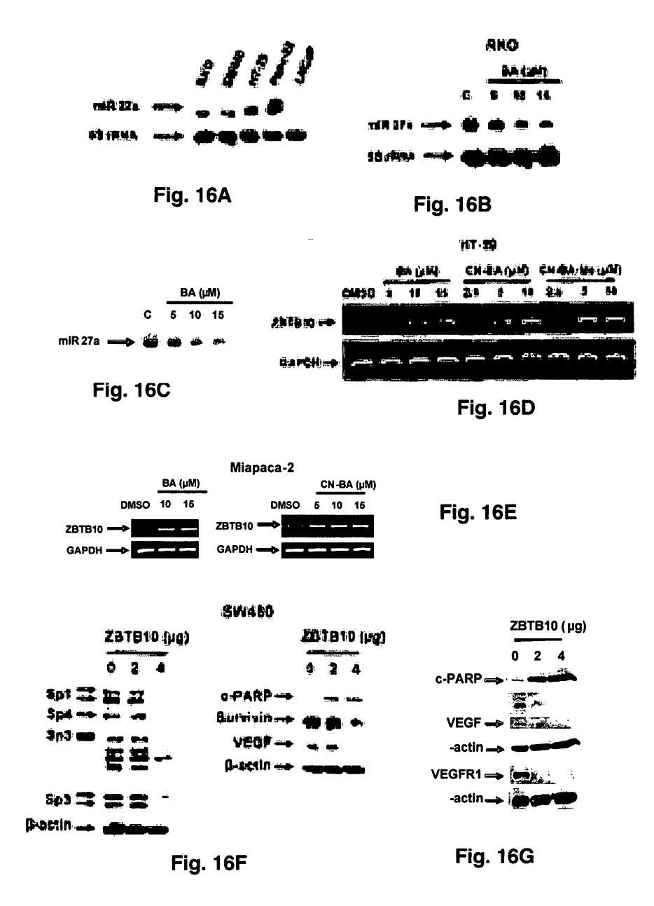

FIG. 16A show that miR27a is expressed in RKO, SW480, HT-29, and Panc-28 colon cancer cells, but not in LNCaP cells. Also, treatment of RKO cells and Panc-28 cells with BA decreases miR-27a in these cell lines (FIG. 16B-16C). It has been demonstrated that miR-27a regulated expression of ZBTB0/R1NZF gene which has been identified as an Sp-suppressor gene. FIGS. 16D-16E show that decreased expression of miR-27a in HT-29 cells treated with BA, CN-BA and CN-BA-Me and in Miapaca-2 cells treated with BA was paralleled by increased expression of ZBTB10.

Since BA decreases mR-27a and increases ZBTB10 expression in RKO cells and since this also is accompanied by BA-induced Sp protein expression (FIG. 15A-15B), the link between miR-27a/ZBTB10 and decreased Sp protein expression was examined. FIGS. 16F-16G show that, like BA, overexpression of ZBTB10 decreases Sp protein expression, decreases survivin and VEGF and induces caspase-dependent PARP cleavage in SW480 colon cancer cells and Miapaca-2 pancreatic cancer cells. This correlates with the lack of miR026a expression in LNCaP cells since BA induces proteasome-dependent degradation of Sp proteins in this cell line (FIGS. 2A-2C).

The following references are cited herein.
1. Pisha et al. Nat. Med. 1:1046-1051, 1995.
2. You et al. Bioorg. Med. Chem Lett. 13: 3137-3140, 2003.
3. Kim et al. Bioorg. Med. Chem Lett. 9:1201-1204, 1999.
4. Abdelrahim et al. Cancer Res. 64:6740-6749, 2004.
5. Abdelrahim et al. J. Natl. Cancer Inst. 98:855-868, 2006.
6. Liao et al. Mol. Cancer Ther, 4:505-515, 2005.
7. Abdelrahim, M. and Safe, S. Mol. Pharmacol. 68:317-329, 2005.
8. Li et al. J. Biol. Chem. 277:19649-19657, 2002.
9. Chintharlapalli et al. Mol. Cancer Therap. In press, 2007.
10. Hong et al. Endocrinology, 145:5774-5785, 2004.
11. Chintharlapalli et al. Cancer Res. 64:5994-6001, 2004.
12. Chintharlapalli et al. Mol. Cancer Therap. 5:1362-1370, 2006.
13. Chintharlapalli et al. Mol. Pharmacol. 68:119-128, 2005.

Any patents or publications mentioned in this specification are indicative of the levels of those skilled in the art to which the invention pertains. These patents and publications are incorporated by reference herein to the same extent as if each individual publication was incorporated by reference specifically and individually. One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A betulinic acid analog or derivative compound having the structural formula: 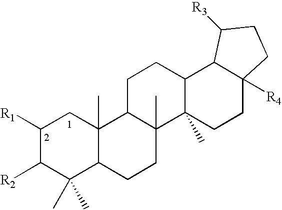
wherein a bond between C1 and C2 is a single bond or a double bond;
R₁ is H, CN, Cl, Br, F, I, CH₃, CF₃, OCH₃, N(CH₃)₂, or phenyl;
R₂ is OH or ═O;
R₃ is COOH, COOC₁₋₄alkyl, COONH₂, COONH(C₁₋₄alkyl), COON(C₁₋₄alkyl)₂, or CHO; and
R₄ is CH(CH₃)₂ or C(CH3)=CH₂; or a pharmacologically effective salt or hydrate thereof.

2. The compound of claim 1, wherein R₁ is Cl, Br or CN.

3. The compound of claim 1, wherein R₂ is ═O.

4. The compound of claim 1, wherein R₃ is COOH or COOCH₃.

5. The compound of claim 1, wherein C1-C2 is a double bond, R₁ is CN, R₂ is ═O and R₃ is COOH or COOCH₃.

6. The compound of claim 1, wherein R₁ is Cl or Br, R₂ is ═O, and R₃ is COOCH₂CH₃ or CHO.

7. A method for inhibiting an activity of one or more specificity protein (Sp) transcription factors in a cell associated with a neoplastic disease, comprising:
decreasing expression of a microRNA in the cell via contact with one or more of betulinic acid or the betulinic acid analog or derivative compounds of claim 1 or a combination thereof thereby increasing expression of an Sp repressor gene in the cell.

8. The method of claim 7, wherein the Sp transcription factor is an Sp1 protein, an Sp3 protein, an Sp4 protein or a combination thereof.

9. The method of claim 7, wherein the microRNA is miR-27a.

10. The method of claim 7, wherein the Sp suppressor gene is ZBTB10.

11. The method of claim 7, wherein the neoplastic disease a kidney cancer, a bladder cancer or prostate cancer, a colon cancer, a melanoma, a pancreatic cancer, or a breast cancer.

12. A method for inhibiting proliferation of cells associated with a neoplastic disease, comprising:
contacting the cell with one or more of betulinic acid or the betulinic acid analog or derivative compounds of claim 1 or a combination thereof.

13. The method of claim 12, wherein a plurality of the cells forms a tumor associated with the neoplastic disease in a subject, the method further comprising:
administering a pharmacologically effective amount of one or more other anticancer drugs to the subject.

14. The method of claim 12, wherein betulinic acid or the analog or derivative thereof is effective to increase expression of Sp suppressor gene ZBTB10, induce degradation of one or more Sp transcription factors in the cells, or to activate PPARγ responses in the cells or a combination thereof.

15. The method of claim 14, wherein the Sp transcription factor is an Sp1 protein, an Sp3 protein, an Sp4 protein or a combination thereof.

16. The method of claim 12, wherein the neoplastic disease a kidney cancer, a bladder cancer or prostate cancer, a colon cancer, a melanoma, a pancreatic cancer, or a breast cancer.

17. A method for treating a cancer in a subject, comprising:
administering a pharmacologically effective amount of one or more of the betulinic acid analog or derivative compounds of claim 1 to the subject, wherein said compound(s) inhibits growth of cancer cells thereby treating the cancer.

18. The method of claim 17, wherein the compound is effective to inhibit angiogenesis in a tumor associated with the cancer or to inhibit metastasis of cancer cells associated with the cancer or both.

19. The method of claim 17, wherein the cancer is a kidney cancer, a bladder cancer or prostate cancer, a colon cancer, a melanoma, a pancreatic cancer, or a breast cancer.

20. A method for reducing toxicity of a cancer therapy in a subject in need thereof, comprising:
administering to the subject a pharmacologically effective amount of one or more of the betulinic acid analogs or derivatives of claim 1 and another anticancer drug, wherein a dosage of the anticancer drug administered with the betulinic acid or an analog or derivative thereof is lower than a dosage required when the anticancer drug is administered singly, thereby reducing toxicity of the cancer therapy to the individual.

21. The method of claim 20, wherein the anticancer drug is administered concurrently or sequentially with the betulinic acid analog or derivative.

22. The method of claim 20, wherein the cancer is a kidney cancer, a bladder cancer or prostate cancer, a colon cancer, a melanoma, a pancreatic cancer, or a breast cancer.

23. A synthetic analog or derivative of betulinic acid having the structural formula: 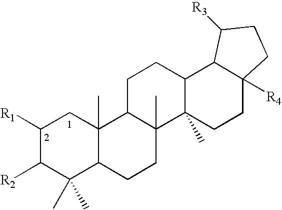
wherein a bond between C1 and C2 is a single bond or a double bond;
R₁ is CN, CH₃, CF₃, OCH₃, N(CH₃)₂, or phenyl;
R₂ is OH or ═O;
R₃ is COOH, COOCH₃, COOCH₂CH₃, or CHO; and
R₄ is CH(CH₃)₂ or C(CH3)=CH₂; or a pharmacologically effective salt or hydrate thereof.

24. The synthetic betulinic acid analog or derivative of claim 23, wherein C1-C2 is a double bond, R₁ is CN, R₂ is ═O and R₃ is COOH or COOCH₃.

25. The synthetic betulinic acid analog or derivative of claim 23, wherein R₃ is COOCH₂CH₃ or CHO, R₁ further comprising Cl, Br, F, or I.


SEQUENCE LISTING (4 sequences)

SEQ ID NO: 1 - DNA, 21 bp, artificial sequence
  primer_binding: KLF4 sense primer
ctatggcagggagtccgctcc

SEQ ID NO: 2 - DNA, 21 bp, artificial sequence
  primer_binding: KLF4 antisense primer
atgaccgacgggctgccgtac

SEQ ID NO: 3 - DNA, 22 bp, artificial sequence
  primer_binding: GADPH sense primer
acggatttggtcgtattgggcg

SEQ ID NO: 4 - DNA, 20 bp, artificial sequence
  primer_binding: GADPH antisense primer
ctcctggaagatggtgatgg